(19) United States
(12) Patent Application Publication
(10) Pub. No.: US 2023/0315904 A1
(43) Pub. Date: Oct. 5, 2023

(54) DIGITAL LEDGER BASED HEALTH DATA SHARING AND MANAGEMENT

(71) Applicants: Victoria LEMIEUX, VANCOUVER (CA); Artemij VOSKOBOJNIKOV, VANCOUVER (CA); Ravneet KAUR, VANCOUVER (CA); Robert FRASER, VANCOUVER (CA); Ian COSTANZO, VANCOUVER (CA)

(72) Inventors: VICTORIA LEMIEUX, VANCOUVER (CA); ARTEMIJ VOSKOBOJNIKOV, VANCOUVER (CA); RAVNEET KAUR, VANCOUVER (CA); ROBERT FRASER, VANCOUVER (CA); IAN COSTANZO, VANCOUVER (CA)

(21) Appl. No.: 18/006,214

(22) PCT Filed: Jul. 20, 2021
(86) PCT No.: PCT/CA2021/051017

§ 371 Date: Jan. 20, 2023

Related U.S. Application Data
(60) Provisional application No. 63/162,719, filed on Mar. 18, 2021.
(60) Provisional application No. 63/053,880, filed on Jul. 20, 2020.

(51) Int. Cl. G06F 21/62; G16H 10/60; H04L 9/00

(52) U.S. Cl. CPC G06F 21/6254; G16H 10/60; H04L 9/50; G06Q 40/08

(57) ABSTRACT

Increased regulation reflects individuals' growing concerns about sharing their health data. But, if left unaddressed, these regulations act as a barrier to healthcare researchers and providers accessing the real-world health data they need for AI-powered health advances. Accordingly, the inventors have established a decentralized ledger based self-sovereign data management platform for individuals. Via the platform an individual is provided a personalized health wallet to supporting self-sovereign data management giving them control and custody of their own health data. The platform provides for data credentialing to ensure quality and support of verifiable claims, e.g., about an individuals' consent to share and/or a third party research project having ethics approval, zero-knowledge proofs to protect individual privacy and promote secure data sharing, and support for privacy-preserving audit, accountability and compliance relating individuals' consent and data sharing.

### FIELD OF THE INVENTION

This application claims the benefit of priority from World Intellectual Property Organization Patent Application PCT/CA2021/051017 filed Jul. 20, 2021; which itself claims the benefit of priority from U.S. Provisional Patent Application 63/162,719 filed Mar. 18, 2021 and claims the benefit of priority from U.S. Provisional Patent Application 63/053,880 filed Jul. 20, 2020; the entire contents of each being herein incorporated by reference.

### FIELD OF THE INVENTION

This patent application relates to personal data and more particularly methods and systems for transferring personal data with a granularity established by the owner of the personal data, establishing self-sovereign identity of the personal data, transferring personal data without releasing the identity of the owner of the personal data, and exploiting a decentralized ledger to establish transfer of personal data where the transfer is performed independent of the decentralized ledger.

### BACKGROUND OF THE INVENTION

Over the past decades the evolution of the Internet, low cost high performance portable electronic devices, and high speed telecommunications, for example, have meant that the storage and use of our personal data has evolved rapidly leading to new issues such as identity theft, theft of personal data and the sale of personal data. Accordingly, there is a requirement for increased security and management of personal data.

For example, personalized artificial intelligence (AI) driven health empowers individuals to take control of their health and has the potential to drive positive health and social benefits. However, it faces many challenges as privacy and security of client data is a major concern, with almost daily news about data breaches and mounting consumer distrust of large, centralized platforms that aggregate data and use it for secondary purposes without individuals' knowledge or consent. As a result, regulations are emerging such as the General Data Protection Regulation (GDPR). Increased regulation reflects individuals' growing concerns about sharing their health data, concerns which, if left unaddressed, can act as a barrier to healthcare researchers and providers accessing the real-world health data they need for AI-powered health advances.

Accordingly, it would be beneficial to provide individuals with a decentralized ledger based self-sovereign data management platform that allows for:
a personalized health wallet to support self-sovereign data management giving customers control and custody of their own health data;
data credentialing to ensure quality and support of verifiable claims, e.g., about individuals' consent to share and third party ethics approval;
zero-knowledge proofs to protect individual privacy & promote secure data sharing; and
support for privacy-preserving audit, accountability and compliance relating individuals' consent and data sharing.

It would be further beneficial for the decentralized ledger based self-sovereign data management platform to support reward systems to incentivize individual data sharing and behavioral change to encourage wellness as well as supporting a business ecosystem for mutually beneficial business partnerships.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to personal data and more particularly methods and systems for transferring personal data with a granularity established by the owner of the personal data, establishing self-sovereign identity of the personal data, transferring personal data without releasing the identity of the owner of the personal data, and exploiting a decentralized ledger to establish transfer of personal data where the transfer is performed independent of the decentralized ledger.

In accordance with an embodiment of the invention there is provided a method of transferring an item of personal data comprising:
establishing and verifying identities of a provider of the item of personal data and a seeker of the item of personal data in dependence upon Decentralized Identifiers of the provider and the seeker stored within one or more blockchains; and
transferring the item of personal data from the provider to the seeker independent of the one or more blockchains.

In accordance with an embodiment of the invention there is provided a method of transferring an item of personal data comprising:
establishing and verifying privacy-preserving decentralized identities of a provider of the item of personal data and a seeker of the item of personal data in dependence upon Public Decentralized Identifiers of the issuer and the seeker stored within one or more blockchains;
transferring the item of personal data from the provider to the seeker independent of the one or more blockchains; wherein
the transfer of the item of personal data is performed without an identity of the provider being exposed to either the seeker or any third party.

In accordance with an embodiment of the invention there is provided a method of transferring an item of personal data comprising:
issuing a credential to a party who becomes the owner of that embodiment of personal data establishing a first Decentralized Identifier (DID) of an owner of the item of personal data;
establishing a second DID of a party seeking to acquire the item of personal data;
establishing a proof of a truth claim in dependence upon verifying the credential held by the first DID by the second DID;
transferring from a first wallet associated with the owner of the personal data the item of personal data to a second wallet associated with another party.

In accordance with an embodiment of the invention there is provided a method comprising:
transferring personal data between an owner of the personal data and an acquirer of the personal data; wherein
the transfer is established by storing and processing non-personal data stored upon a blockchain; and
the actual transfer of the personal data is performed independent of the blockchain.

In accordance with an embodiment of the invention there is provided a method comprising:
generating by an issuer a consent receipt identity and issuing to a holder a consent enablement credential;
establishing whether the holder accepts the consent enablement credential;
upon a positive determination of the holder accepting the consent enablement credential transmitting an initial acknowledgement from the holder to the issuer;
sending to the holder from the issuer a consent proof request in dependence upon receipt of the initial acknowledgement;
establishing whether the holder accepts the consent proof request;
upon a positive determination of the holder accepting the consent proof request transmitting a consent proof presentation from the holder to the issuer;
establishing whether the issuer accepts the consent proof presentation received from the holder;
upon a positive determination of the issuer accepting the consent proof presentation sending proof data to a proof registry from the issuer.

In accordance with an embodiment of the invention there is provided a method comprising:
establishing a request for a biomarker from a holder of the biomarker to a distributed ledger software application;
generating with the distributed ledger software application shares S_{I}, S_{R}, and S_{H} in dependence upon receipt of the request for the biomarker;
generating with the distributed ledger software application a credential relating to the biomarker requested;
transmitting the biomarker identity and shares S_{R} and S_{H} to the holder;
determining whether the holder accepts the biomarker identity and shares S_{R} and S_{H};
upon a positive determination of the holder accepting the biomarker identity and shares S_{R} and S_{H} transmitting the biomarker identity and share S_{R} to a researcher seeking access to the biomarker of the holder;
generating a consent receipt identity upon receipt of the biomarker identity and share S_{R};
issuing and transmitting a consent enablement credential to the holder from the researcher;
determining whether the holder accepts the consent enablement credential;
upon a positive determination of the holder accepting the consent enablement credential transmitting an initial acknowledgement to the researcher;
upon receipt by the researcher of the initial acknowledgement generating a consent proof request comprising the share S_{R} and transmitting the consent proof request to a proof registry and the holder;
storing the consent proof request within the proof registry;
determining whether the holder accepts the consent proof request;
upon a positive determination of the holder accepting the consent proof request generating a consent proof presentation and transmitting the consent proof presentation to the researcher.

In accordance with an embodiment of the invention there is provided a method comprising:
receiving by a proof registry a request for consent data from an auditor;
determining upon the proof registry whether to accept the request; upon a positive determination retrieving the consent data from a database associated with the proof registry;
transmitting a biomarker identity and a share S_{R} from the proof registry to the auditor;
regenerating by the auditor a secret;
transmitting the secret, biomarker identity and share to a distributed ledger software application;
mapping with the distributed ledger software application a share in dependence upon the biomarker identity and retrieving from another database associated with the distributed ledger software application another share S_{I};
reconstructing with the distributed ledger software application a hash in dependence upon the share S_{R} and another share S_{I};
retrieving an identity through a lookup in dependence upon the hash from a further database associated with the distributed ledger software application; and
transmitting the retrieved identity to the auditor.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts an exemplary network environment within which configurable electrical devices according to and supporting embodiments of the invention may be deployed and operate; and

FIG. 2 depicts an exemplary wireless portable electronic device supporting communications to a network such as depicted in FIG. 1 and configurable electrical devices according to and supporting embodiments of the invention;

FIG. 3 depicts an exemplary schematic of a high level architecture of an architecture exploiting transactions upon a blockchain network according to an embodiment of the invention for private and secure health data management and sharing;

FIG. 4 depicts an exemplary architecture for front-end and back-end services of a blockchain framework supporting Decentralized Ledger Personal Data (DeLePeDa) Systems, Applications and Platforms (DeLePeDa-SAPs) according to embodiments of the invention;

FIG. 5 depicts a high level architecture of a blockchain framework supporting DeLePeDa-SAPs according to embodiments of the invention;

FIG. 6 depicts a logical process flow and architecture for a first use case of DeLePeDa-SAPs according to embodiments of the invention;

FIG. 7 depicts a logical process flow and architecture for a second use case of DeLePeDa-SAPs according to embodiments of the invention;

FIG. 8 depicts an exemplary schema for requests and data flow for the second use case of DeLePeDa -SAPs according to embodiments of the invention;

FIG. 9 depicts an exemplary data architecture employed by DeLePeDa -SAPs according to embodiments of the invention;

FIG. 10 depicts historical and self-sovereign identity loci of control wherein the self-sovereign locus of control is supported by DeLePeDa -SAPs according to embodiments of the invention;

FIG. 11 depicts an exemplary process flow between parties and a blockchain within DeLePeDa-SAPs according to embodiments of the invention;

FIG. 12 depicts an exemplary process flow between parties and a blockchain for an initial user request to obtain personal data stored within a wallet of another party;

FIG. 13 depicts an exemplary Business Process Modeling Notation (BPMN) diagram for an audit data registry (also known as an audit data repository) according to an embodiment of the invention;

FIG. 14 depicts an exemplary BPMN diagram for an audit data registry according to an embodiment of the invention;

FIG. 15 depicts re-verification of a proof for an audit data registry according to an embodiment of the invention;

FIG. 16 depicts a credential as stored within an audit data registry according to an embodiment of the invention;

FIG. 17 depicts a proof as stored within an audit data registry according to an embodiment of the invention;

FIG. 18 depicts a logical process flow and architecture for a DeLePeDa -SAP according to embodiments of the invention;

FIG. 19 depicts an exemplary architecture for a for a DeLePeDa -SAP according to embodiments of the invention;

FIG. 20 depicts an exemplary process flow for a researcher project set up and research ethics board certification exploiting DeLePeDa-SAPs according to embodiments of the invention;

FIG. 21 depicts an exemplary process flow for a user connection and credential receipt within DeLePeDa-SAPs according to embodiments of the invention;

FIG. 22 depicts an exemplary process flow for project eligibility proof within DeLePeDa-SAPs according to embodiments of the invention;

FIG. 23 depicts an exemplary process flow for a client receiving consent credentials from a research within DeLePeDa-SAPs according to embodiments of the invention;

FIG. 24 depicts an exemplary process flow for a client consenting with proof and sharing health data with proof within DeLePeDa-SAPs according to embodiments of the invention;

FIG. 25 depicts an exemplary process flow for consent receipt within DeLePeDa-SAPs according to embodiments of the invention;

FIG. 26 depicts exemplary screenshots of a software application depicting the consent receipt and proofs as described within FIG. 25;

FIG. 27 depicts exemplary interactions within DeLePeDa-SAPs according to embodiments of the invention;

FIG. 28 depicts an exemplary process flow for issuing health credentials within DeLePeDa -SAPs according to embodiments of the invention;

FIG. 29 depicts an exemplary process flow for a client browsing projects and choosing to enroll within DeLePeDa-SAPs according to embodiments of the invention;

FIG. 30 depicts an exemplary process flow for client to researcher interactions for sharing data with consent within DeLePeDa-SAPs according to embodiments of the invention;

FIG. 31 depicts an exemplary SSI architecture within DeLePeDa-SAPs according to embodiments of the invention;

FIG. 32 depicts an exemplary architecture with messaging flow for project setup and research ethics board certification within DeLePeDa-SAPs according to embodiments of the invention;

FIG. 33 depicts an exemplary architecture with messaging flow for client connection and credential receipt within DeLePeDa-SAPs according to embodiments of the invention;

FIG. 34 depicts an exemplary architecture with messaging flow for project eligibility proof within DeLePeDa -SAPs according to embodiments of the invention;

FIG. 35 depicts an exemplary architecture with messaging flow for consent credential receipt from a researcher within DeLePeDa-SAPs according to embodiments of the invention;

FIG. 36 depicts an exemplary architecture with messaging flow for providing consent and sharing health data (both with proof) within DeLePeDa-SAPs according to embodiments of the invention;

FIG. 37 depicts an exemplary BPMN diagram for a handshake with respect to a holder and a researcher (requester) relating to providing data relating to the holder to the researcher according to an embodiment of the invention; and

FIG. 38 depicts an exemplary BPMN diagram for retrieval of identity information relating to a consent by an auditor from an audit data registry according to an embodiment of the invention.

### DETAILED DESCRIPTION

The present invention is directed to personal data and more particularly methods and systems for transferring personal data with a granularity established by the owner of the personal data, establishing self-sovereign identity of the personal data, transferring personal data without releasing the identity of the owner of the personal data, and exploiting a decentralized ledger to establish transfer of personal data where the transfer is performed independent of the decentralized ledger.

The ensuing description provides representative embodiment(s) only, and is not intended to limit the scope, applicability, or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may”, “might”, “can” or “could” be included, that particular component, feature, structure, or characteristic is not required to be included.

Reference to terms such as “left”, “right”, “top”, “bottom”, “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including”, “comprising”, “consisting” and grammatical variants thereof do not preclude the addition of one or more components, features, steps, integers, or groups thereof and that the terms are not to be construed as specifying components, features, steps, or integers. Likewise, the phrase “consisting essentially of”, and grammatical variants thereof, when used herein is not to be construed as excluding additional components, steps, features integers or groups thereof but rather that the additional features, integers, steps, components, or groups thereof do not materially alter the basic and novel characteristics of the claimed composition, device, or method. If the specification or claims refer to “an additional” element, that does not preclude there being more than one of the additional element.

A “record” as used herein and throughout this disclosure, refer to, but is not limited to, information created, received, and maintained as evidence and as an asset by an organization or person, in pursuit of legal obligations or in the transaction of business.

A “transaction record” as used herein and throughout this disclosure, refer to, but is not limited to, a record documenting a transaction.

A “ledger record” or “ledger records” as used herein and throughout this disclosure, refer to, but is not limited to, a record or records containing one or more of transaction record(s), hash value(s) of transaction record(s), and reference(s) to transaction record(s) recorded on one or more distributed ledgers (e.g. blockchain) using a distributed ledger technology (DLT).

A “record system” or “records system” as used herein and throughout this disclosure, refer to, but is not limited to, an information system that captures, manages, and provides access to records over time.

A “credential” as used herein and throughout this disclosure, refers to, but is not limited to, a set of claims made by an issuer.

A “verifiable credential” as used herein and throughout this disclosure, refers to, but is not limited to, a tamper-evident credential that has an authorship that can be cryptographically verified.

An “issuer” as used herein and throughout this disclosure, refers to, but is not limited to, a role an entity can perform by asserting claims about one or more subjects, creating a verifiable credential from these claims, and transmitting the verifiable credential to a holder.

A “holder” as used herein and throughout this disclosure, refers to, but is not limited to, a role an entity might perform by possessing one or more verifiable credentials and generating presentations from them.

A “verifier” as used herein and throughout this disclosure, refers to, but is not limited to, an entity verifying a claim about a given subject.

A “proof request” as used herein and throughout this disclosure, refers to, but is not limited to, a request for one or more verifiable credentials, issued by one or more issuers, held by one or more holders.

A “proof presentation” as used herein and throughout this disclosure, refers to, but is not limited to, data derived from one or more verifiable credentials, issued by one or more issuers, that is shared with a specific verifier or verifiers.

A “proof verification” as used herein and throughout this disclosure, refers to, but is not limited to, an evaluation as to whether a verifiable credential or verifiable presentation is an authentic and timely statement of the issuer or presenter, respectively.

A “wireless standard” as used herein and throughout this disclosure, refers to, but is not limited to, a standard for transmitting signals and/or data through electromagnetic radiation which may be optical, radio-frequency (RF) or microwave although typically RF wireless systems and techniques dominate. A wireless standard may be defined globally, nationally, or specific to an equipment manufacturer or set of equipment manufacturers. Dominant wireless standards at present include, but are not limited to IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, Bluetooth, Wi-Fi, Ultra-Wideband and WiMAX. Some standards may be a conglomeration of sub-standards such as IEEE 802.11 which may refer to, but is not limited to, IEEE 802.1a, IEEE 802.11b, IEEE 802.11g, or IEEE 802.11n as well as others under the IEEE 802.11 umbrella.

A “wired standard” as used herein and throughout this disclosure, generally refers to, but is not limited to, a standard for transmitting signals and/or data through an electrical cable discretely or in combination with another signal. Such wired standards may include, but are not limited to, digital subscriber loop (DSL), Dial-Up (exploiting the public switched telephone network (PSTN) to establish a connection to an Internet service provider (ISP)), Data Over Cable Service Interface Specification (DOCSIS), Ethernet, Gigabit home networking (G.hn), Integrated Services Digital Network (ISDN), Multimedia over Coax Alliance (MoCA), and Power Line Communication (PLC, wherein data is overlaid to AC/DC power supply). In some embodiments a “wired standard” may refer to, but is not limited to, exploiting an optical cable and optical interfaces such as within Passive Optical Networks (PONs) for example.

A “sensor” as used herein may refer to, but is not limited to, a transducer providing an electrical output generated in dependence upon a magnitude of a measure and selected from the group comprising, but is not limited to, environmental sensors, medical sensors, biological sensors, chemical sensors, ambient environment sensors, position sensors, motion sensors, thermal sensors, infrared sensors, visible sensors, RFID sensors, and medical testing and diagnosis devices.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device, and an electronic reader.

A “fixed electronic device” (FED) as used herein and throughout this disclosure, refers to a wireless and/or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

A “server” as used herein, and throughout this disclosure, refers to one or more physical computers co-located and/or geographically distributed running one or more services as a host to users of other computers, PEDs, FEDs, etc. to serve the client needs of these other users. This includes, but is not limited to, a database server, file server, mail server, print server, web server, gaming server, or virtual environment server.

An “application” (commonly referred to as an “app”) as used herein may refer to, but is not limited to, a “software application”, an element of a “software suite”, a computer program designed to allow an individual to perform an activity, a computer program designed to allow an electronic device to perform an activity, and a computer program designed to communicate with local and/or remote electronic devices. An application thus differs from an operating system (which runs a computer), a utility (which performs maintenance or general-purpose chores), and a programming tools (with which computer programs are created). Generally, within the following description with respect to embodiments of the invention an application is generally presented in respect of software permanently and/or temporarily installed upon a PED and/or FED.

An “enterprise” as used herein may refer to, but is not limited to, a provider of a service and/or a product to a user, customer, or consumer. This includes, but is not limited to, a retail outlet, a store, a market, an online marketplace, a manufacturer, an online retailer, a charity, a utility, and a service provider. Such enterprises may be directly owned and controlled by a company or may be owned and operated by a franchisee under the direction and management of a franchiser.

A “service provider” as used herein may refer to, but is not limited to, a third party provider of a service and/or a product to an enterprise and/or individual and/or group of individuals and/or a device comprising a microprocessor. This includes, but is not limited to, a retail outlet, a store, a market, an online marketplace, a manufacturer, an online retailer, a utility, an own brand provider, and a service provider wherein the service and/or product is at least one of marketed, sold, offered, and distributed by the enterprise solely or in addition to the service provider.

A “third party” or “third party provider” as used herein may refer to, but is not limited to, a so-called “arm's length” provider of a service and/or a product to an enterprise and/or individual and/or group of individuals and/or a device comprising a microprocessor wherein the consumer and/or customer engages the third party but the actual service and/or product that they are interested in and/or purchase and/or receive is provided through an enterprise and/or service provider.

A “user” as used herein may refer to, but is not limited to, an individual or group of individuals. This includes, but is not limited to, private individuals, employees of organizations and/or enterprises, members of community organizations, members of charity organizations, men, and women. In its broadest sense the user may further include, but not be limited to, software systems, mechanical systems, robotic systems, android systems, etc. that may be characterised by an ability to exploit one or more embodiments of the invention. A user may also be associated through one or more accounts and/or profiles with one or more of a service provider, third party provider, enterprise, social network, social media etc. via a dashboard, web service, website, software plug-in, software application, and graphical user interface.

“Biometric” information as used herein may refer to, but is not limited to, data relating to a user characterised by data relating to a subset of conditions including, but not limited to, their environment, medical condition, biological condition, physiological condition, chemical condition, ambient environment condition, position condition, neurological condition, drug condition, and one or more specific aspects of one or more of these said conditions. Accordingly, such biometric information may include, but not be limited, blood oxygenation, blood pressure, blood flow rate, heart rate, temperate, fluidic pH, viscosity, particulate content, solids content, altitude, vibration, motion, perspiration, EEG, ECG, energy level, etc. In addition, biometric information may include data relating to physiological characteristics related to the shape and/or condition of the body wherein examples may include, but are not limited to, fingerprint, facial geometry, baldness, DNA, hand geometry, odour, and scent. Biometric information may also include data relating to behavioral characteristics, including but not limited to, typing rhythm, gait, and voice.

“User information” as used herein may refer to, but is not limited to, user behavior information and/or user profile information. It may also include a user's biometric information, an estimation of the user's biometric information, or a projection/prediction of a user's biometric information derived from current and/or historical biometric information.

A “wearable device” or “wearable sensor” relates to miniature electronic devices that are worn by the user including those under, within, with or on top of clothing and are part of a broader general class of wearable technology which includes “wearable computers” which in contrast are directed to general or special purpose information technologies and media development. Such wearable devices and/or wearable sensors may include, but not be limited to, smartphones, smart watches, e-textiles, smart shirts, activity trackers, smart glasses, environmental sensors, medical sensors, biological sensors, physiological sensors, chemical sensors, ambient environment sensors, position sensors, neurological sensors, drug delivery systems, medical testing and diagnosis devices, and motion sensors.

An “item of apparel” as used herein refers to an item of clothing worn by a user typically made of a fabric, fabrics, a textile, or textiles. A wearable device or wearable sensor may form an integral part of an item of apparel or be demountable attachable to an item of apparel.

“Electronic content” (also referred to as “content” or “digital content”) as used herein may refer to, but is not limited to, any type of content that exists in the form of digital data as stored, transmitted, received and/or converted wherein one or more of these steps may be analog although generally these steps will be digital. Forms of digital content include, but are not limited to, information that is digitally broadcast, streamed, or contained in discrete files. Viewed narrowly, types of digital content include popular media types such as MP3, JPG, AVI, TIFF, AAC, TXT, RTF, HTML, XHTML, PDF, XLS, SVG, WMA, MP4, FLV, and PPT, for example, as well as others, see for example http://en.wikipedia.org/wiki/List_of_file_formats. Within a broader approach digital content mat include any type of digital information, e.g. digitally updated weather forecast, a GPS map, an eBook, a photograph, a video, a Vine™, a blog posting, a Facebook™ posting, a Twitter™ tweet, online TV, etc. The digital content may be any digital data that is at least one of generated, selected, created, modified, and transmitted in response to a user request, said request may be a query, a search, a trigger, an alarm, and a message for example.

A “profile” as used herein, and throughout this disclosure, refers to a computer and/or microprocessor readable data file comprising data relating to settings and/or limits of an adult device. Such profiles may be established by a manufacturer/supplier/provider of a device, service, etc. or they may be established by a user through a user interface for a device, a service, or a PED/FED in communication with a device, another device, a server, or a service provider etc.

A “computer file” (commonly known as a file) as used herein, and throughout this disclosure, refers to a computer resource for recording data discretely in a computer storage device, this data being electronic content. A file may be defined by one of different types of computer files, designed for different purposes. A file may be designed to store electronic content such as a written message, a video, a computer program, or a wide variety of other kinds of data. Some types of files can store several types of information at once. A file can be opened, read, modified, copied, and closed with one or more software applications an arbitrary number of times. Typically, files are organized in a file system which can be used on numerous different types of storage device exploiting different kinds of media which keeps track of where the files are located on the storage device(s) and enables user access. The format of a file is defined by its content since a file is solely a container for data, although, on some platforms the format is usually indicated by its filename extension, specifying the rules for how the bytes must be organized and interpreted meaningfully. For example, the bytes of a plain text file are associated with either ASCII or UTF-8 characters, while the bytes of image, video, and audio files are interpreted otherwise. Some file types also allocate a few bytes for metadata, which allows a file to carry some basic information about itself

“Encryption” as used herein may refer to, but is not limited to, the processes of encoding messages or information in such a way that only authorized parties can read it. This includes, but is not limited to, symmetric key encryption through algorithms such as Twofish, Serpent, AES (Rijndael), Blowfish, CASTS, RC4, 3DES, and IDEA for example, and public-key encryption through algorithms such as Diffie-Hellman, Digital Signature Standard, Digital Signature Algorithm, ElGamal, elliptic-curve techniques, password-authenticated key agreement techniques, Paillier cryptosystem, RSA encryption algorithm, Cramer-Shoup cryptosystem, and YAK authenticated key agreement protocol.

A “blockchain” (originally block chain) represents an embodiment of a cryptographic distributed ledger or an embodiment of a DLT. A blockchain as used herein may refer to, but not be limited to, a continuously growing list of records, called blocks, which are linked and secured using cryptography. Each block typically contains a cryptographic hash of one or more other blocks in the chain, a timestamp and transaction data. By design, a blockchain is inherently resistant to modification of the data and provides for an open, distributed ledger that can record transactions between two parties efficiently and in a verifiable and permanent way. For use as a distributed ledger, a blockchain is typically managed by a peer-to-peer network collectively adhering to a protocol for inter-node communication and validating new blocks. Once recorded, the data in any given block cannot be altered retroactively without the alteration of all subsequent blocks in the chain, which requires the collusion of the network majority. Accordingly, a blockchain is secure by design and exemplifies a distributed computing system with high Byzantine fault tolerance. Decentralized consensus has therefore been achieved with a blockchain which makes them suitable for the recording of events, medical records, and other records management activities, such as identity management, financial transaction processing, documenting provenance, food traceability, voting, etc. Within embodiments of the invention the cryptographic hash may also include a pointer (and possibly a hash) of the address of the next block in the chain.

A “distributed ledger” as used herein may refer to, but not be limited to, a is a database that is consensually shared and synchronized across one or more networks spread across multiple sites, institutions and/or geographies. It allows transactions to have public “witnesses,” thereby making a cyberattack more difficult. The participant at each node of the network can access the recordings shared across that network and can own an identical copy of it. Further, any changes or additions made to the ledger are reflected and copied to all participants quickly, usually within seconds or minutes. Underlying a distributed ledger technology are blockchains.

A “cryptographic currency” or “crypto currency” as used herein may refer to, but not be limited to, a digital asset designed to work as a medium of exchange that uses cryptography to secure its transactions, to control the creation of additional units, and to verify the transfer of assets. Cryptocurrencies are a type of digital currencies, alternative currencies, and virtual currencies. Cryptocurrencies use decentralized control as opposed to centralized electronic money and central banking systems. The decentralized control of each cryptocurrency works through a blockchain, which is a public transaction database, functioning as a distributed ledger.

“Self-Sovereign Identity” (SSI) as used herein may refer to, but not be limited to, a user's digital identity which allows the user to fully create and control their credential(s), without being forced to request permission of an intermediary or centralized authority and gives control over how to the user of how their personal data is shared and used. With an SSI a user has a means of generating and controlling unique identifiers as well as some facility to store identity data. An SSI may be a discrete identity stored in a decentralized manner or a decentralized identity although in its broadest it may, for example, a set of data associated with the user such as social media account data of the user, a history of transactions on an electronic commerce (e-commerce) site by the user, or a set of attestations from other individuals and/or enterprises (e.g. friends or colleagues). Accordingly, in a decentralized identity paradigm of which the SSI forms part the user is at the centre of the framework and there is no need for third parties to issue and administer an identity. However, the user may elect to have its SSI stored by a third party provider or service provider.

Decentralized Ledger Personal Data System and Platform

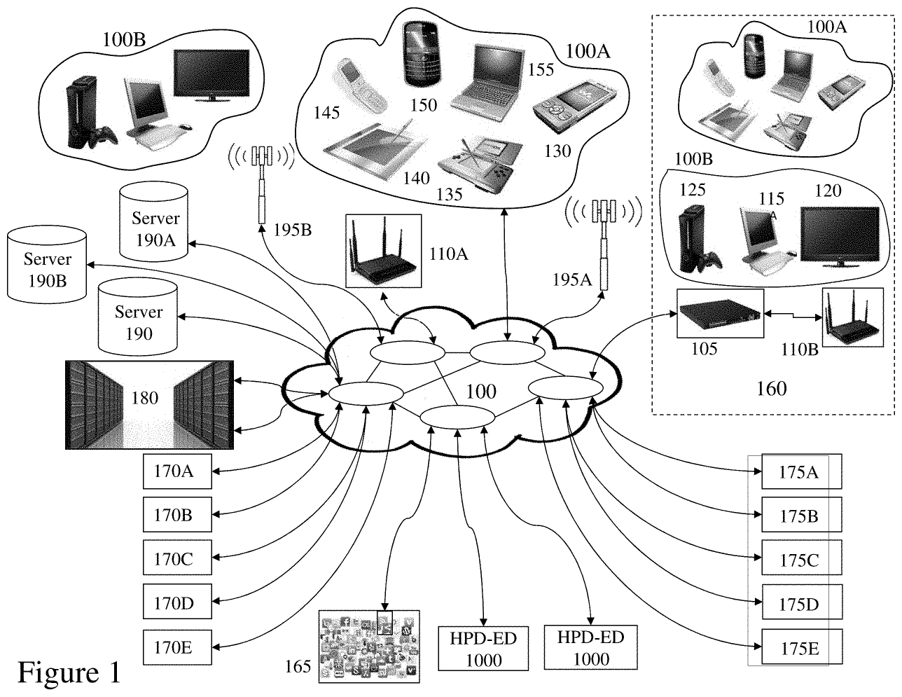

Referring to FIG. 1 there is depicted a Network 100 supporting embodiments of the invention through Decentralized Ledger Personal Data (DeLePeDa) Systems, Applications and Platforms (DeLePeDa-SAPs) according to embodiments of the invention. As shown first and second user groups 100A and 100B respectively interface to a telecommunications Network 100. Within the representative telecommunication architecture, a remote central exchange 180 communicates with the remainder of a telecommunication service providers network via the Network 100 which may include for example long-haul OC-48/OC-192 backbone elements, an OC-48 wide area network (WAN), a Passive Optical Network, and a Wireless Link. The central exchange 180 is connected via the Network 100 to local, regional, and international exchanges (not shown for clarity) and therein through Network 100 to first and second cellular APs 195A and 195B respectively which provide Wi-Fi cells for first and second user groups 100A and 100B, respectively. Also connected to the Network 100 are first and second Wi-Fi nodes 110A and 110B, the latter of which being coupled to Network 100 via router 105. Second Wi-Fi node 110B is associated with commercial service provider 160 comprising other first and second user groups 100A and 100B. Second user group 100B may also be connected to the Network 100 via wired interfaces including, but not limited to, DSL, Dial-Up, DOCSIS, Ethernet, G.hn, ISDN, MoCA, PON, and Power line communication (PLC) which may or may not be routed through a router such as router 105.

Within the cell associated with first AP 110A the first group of users 100A may employ a variety of PEDs including for example, laptop computer 155, portable gaming console 135, tablet computer 140, smartphone 150, cellular telephone 145 as well as portable multimedia player 130. Within the cell associated with second AP 110B are the second group of users 100B which may employ a variety of FEDs including for example gaming console 125, personal computer 115 and wireless/Internet enabled television 120 as well as cable modem 105. First and second cellular APs 195A and 195B respectively provide, for example, cellular GSM (Global System for Mobile Communications) telephony services as well as 3G and 4G evolved services with enhanced data transport support. Second cellular AP 195B provides coverage in the exemplary embodiment to first and second user groups 100A and 100B. Alternatively the first and second user groups 100A and 100B may be geographically disparate and access the Network 100 through multiple APs, not shown for clarity, distributed geographically by the network operator or operators. First cellular AP 195A as show provides coverage to first user group 100A and environment 170, which comprises second user group 100B as well as first user group 100A. Accordingly, the first and second user groups 100A and 100B may according to their particular communications interfaces communicate to the Network 100 through one or more wireless communications standards such as, for example, IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, and IMT-1000. It would be evident to one skilled in the art that many portable and fixed electronic devices may support multiple wireless protocols simultaneously, such that for example a user may employ GSM services such as telephony and SMS and Wi-Fi/WiMAX data transmission, VOIP and Internet access. Accordingly, portable electronic devices within first user group 100A may form associations either through standards such as IEEE 802.15 and Bluetooth as well in an ad-hoc manner.

Also connected to the Network 100 are Social Networks (SOCNETS) 165, first and second service providers 170A and 170B respectively, first and second third party service providers 170C and 170D respectively, and a user 170E. Also connected to the Network 100 are first and second enterprises 175A and 175B respectively, first and second organizations 175C and 175D respectively, and a government entity 175E. Each of these may exploit and/or support DeLePaDa-SAPs which are or support embodiments of the invention. Also depicted are first and second servers 190A and 190B which may host according to embodiments of the inventions multiple services associated with a provider of Decentralized Ledger Personal Data (DeLePeDa) Systems, Applications and Platforms (DeLePeDa-SAPs), contact management systems and contact management applications/platforms; a provider of a SOCNET or Social Media (SOME) exploiting DeLePeDa-SAP features; a provider of a SOCNET and/or SOME not exploiting DeLePeDa-SAP features; a provider of services to PEDS and/or FEDS; a provider of one or more aspects of wired and/or wireless communications; an Enterprise 160 exploiting DeLePeDa-SAP features; license databases; content databases; image databases; content libraries; customer databases; websites; and software applications for download to or access by FEDs and/or PEDs exploiting and/or hosting DeLePeDa-SAP features as well as a user exploiting DeLePeDa-SAPs. First and second primary content servers 190A and 190B may also host for example other Internet services such as a search engine, financial services, third party applications and other Internet based services.

Also depicted in FIG. 1 are DeLePeDa Electronic Devices (DeLePeDa-EDs) 1000 according to embodiments of the invention which allow user, organizations, etc. to access and exploit DeLePeDa-SAPs As depicted in FIG. 1 the DeLePeDa-EDs 1000 can communicate directly to the Network 100. The DeLePeDa-EDs 1000 may communicate to the Network 100 through one or more wireless or wired interfaces included those, for example, selected from the group comprising IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, DSL, Dial-Up, DOCSIS, Ethernet, G.hn, ISDN, MoCA, PON, and Power line communication (PLC).

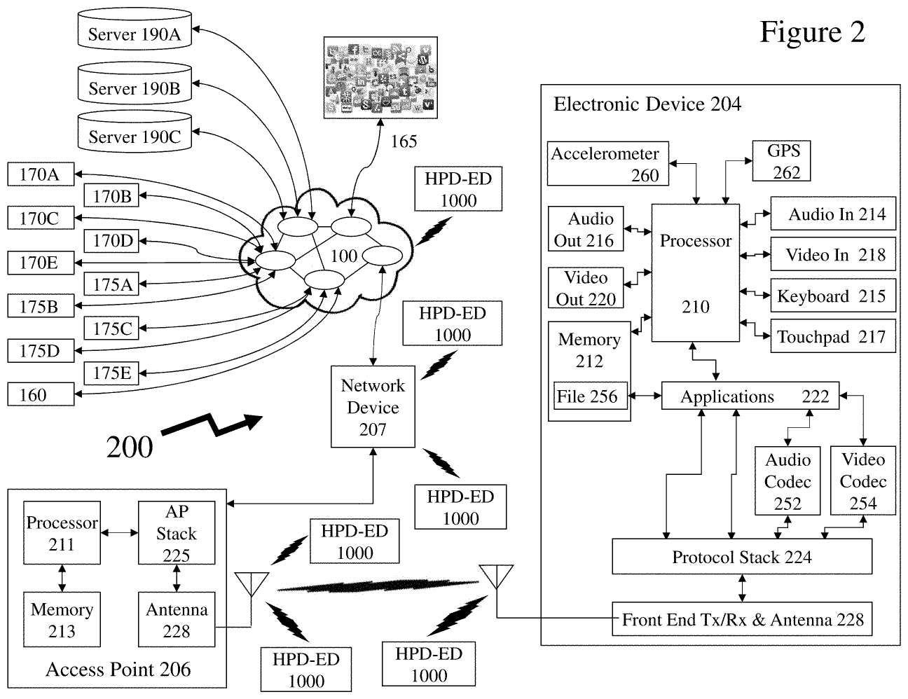

Accordingly, a user may exploit a DeLePeDa-ED 1000 directly to interact with one or more of a PED, a FED, Enterprise 160, SOCNETS 165, first service provider 170A, second service provider 170B, first third party service provider 170C, second third party service provider 170D, and another user 170E. Alternatively, the user may exploit the DeLePeDa-ED 1000 to interact with one or more of a first enterprise 175A, second enterprise 175B, first organization 175C, second organization 175D, a government entity 175E, first server 190A and second server 190B. The user may exploit the DeLePeDa-ED 1000 to perform one or more operations such as accessing/downloading an application which provides DeLePeDa-SAP features according to embodiments of the invention; execute an application already installed providing DeLePeDa-SAP features; execute a web based application providing DeLePeDa-SAP features; access content; generate content; acquire data; and provide data. Similarly, a user may undertake such actions or others exploiting embodiments of the invention by employing the DeLePeDa-ED 1000 in conjunction with a PED or FED within first and second user groups 100A and 100B respectively via one of first and second cellular APs 195A and 195B respectively and first Wi-Fi nodes 110A. It would also be evident that a user may, via exploiting Network 100 communicate via telephone, fax, email, SMS, social media, etc. or trigger one or more actions with respect to a local PED or FED and/or a remote PED or FED using a DeLePeDa-ED 1000. Further, as evident and described below with respect to FIG. 2 the user may trigger one or more actions with respect to another DeLePeDa-ED 1000 from their DeLePeDa-ED 1000 either directly or indirectly.

Now referring to FIG. 2 there is depicted an Electronic Device 204 and network access point 207 supporting DeLePeDa-SAP features according to embodiments of the invention. The Electronic Device 204 may be an DeLePeDa-ED 1000 according to embodiments of the invention or it may interface with an DeLePeDa-ED 1000 according to embodiments of the invention. Electronic Device 204 may include additional elements above and beyond those described and depicted or it may omit some elements described and depicted. Also depicted within the Electronic Device 204 is a protocol architecture as part of a simplified functional diagram of a system 200 that includes an Electronic Device 204, an access point (AP) 206, such as first AP 110, and one or more network devices 207, such as communication servers, streaming media servers, and routers for example such as first and second servers 190A and 190B respectively. Network devices 207 may be coupled to AP 206 via any combination of networks, wired, wireless and/or optical communication links such as discussed above in respect of FIG. 1 as well as directly as indicated. Network devices 207 are coupled to Network 100 and therein Social Networks (SOCNETS) 165, first and second service providers 170A and 170B respectively, first and second third party service providers 170C and 170D respectively, a user 170E, first and second enterprises 175A and 175B respectively, first and second organizations 175C and 175D respectively, and a government entity 175E.

The Electronic Device 204 includes one or more processors 210 and a memory 212 coupled to processor(s) 210. AP 206 also includes one or more processors 211 and a memory 213 coupled to processor(s) 210. A non-exhaustive list of examples for any of processors 210 and 211 includes a central processing unit (CPU), a digital signal processor (DSP), a reduced instruction set computer (RISC), a complex instruction set computer (CISC) and the like. Furthermore, any of processors 210 and 211 may be part of application specific integrated circuits (ASICs) or may be a part of application specific standard products (ASSPs). A non-exhaustive list of examples for memories 212 and 213 includes any combination of the following semiconductor devices such as registers, latches, ROM, EEPROM, flash memory devices, non-volatile random access memory devices (NVRAM), SDRAM, DRAM, double data rate (DDR) memory devices, SRAM, universal serial bus (USB) removable memory, and the like.

Electronic Device 204 may include an audio input element 214, for example a microphone, and an audio output element 216, for example, a speaker, coupled to any of processors 210. Electronic Device 204 may include a video input element 218, for example, a video camera or camera, and a video output element 220, for example an LCD display, coupled to any of processors 210. Electronic Device 204 also includes a keyboard 215 and touchpad 217 which may for example be a physical keyboard and touchpad allowing the user to enter content or select functions within one of more applications 222. Alternatively, the keyboard 215 and touchpad 217 may be predetermined regions of a touch sensitive element forming part of the display within the Electronic Device 204. The one or more applications 222 that are typically stored in memory 212 and are executable by any combination of processors 210. Electronic Device 204 also includes accelerometer 260 providing three-dimensional motion input to the process 210 and GPS 262 which provides geographical location information to processor 210.

Electronic Device 204 includes a protocol stack 224 and AP 206 includes an AP stack 225. Within system 200 protocol stack 224 may be, for example, an IEEE 802.11 protocol stack but alternatively the Electronic Device 204 may exploit other protocol stacks such as an Internet Engineering Task Force (IETF) multimedia protocol stack for example. Elements of protocol stack 224 and AP stack 225 may be implemented in any combination of software, firmware and/or hardware. For example, protocol stack 224 may include an IEEE 802.11-compatible PHY module which is coupled to one or more Front-End Tx/Rx & Antenna 228, an IEEE 802.11-compatible MAC module coupled to an IEEE 802.2-compatible LLC module. Protocol stack 224 may also include a network layer IP module, a transport layer User Datagram Protocol (UDP) module and a transport layer Transmission Control Protocol (TCP) module. Protocol stack 224 may also include a session layer Real Time Transport Protocol (RTP) module, a Session Announcement Protocol (SAP) module, a Session Initiation Protocol (SIP) module and a Real Time Streaming Protocol (RTSP) module. Protocol stack 224 may also include a presentation layer media negotiation module and a call control module as well as one or more audio codecs 252 and one or more video codecs 254 which are depicted separately in this instance. Applications 222 may be able to create maintain and/or terminate communication sessions with any of devices 207 by way of AP 206. Typically, applications 222 may activate any of the SAP, SIP, RTSP, media negotiation and call control modules for that purpose. Typically, information may propagate from the SAP, SIP, RTSP, media negotiation and call control modules to PHY module through TCP module, IP module, LLC module and MAC module.

It would be apparent to one skilled in the art that elements of the Electronic Device 204 may also be implemented within the AP 206 including but not limited to one or more elements of the protocol stack 224, including for example an IEEE 802.11-compatible PHY module, an IEEE 802.11-compatible MAC module, and an IEEE 802.2-compatible LLC module. The AP 206 may additionally include a network layer IP module, a transport layer User Datagram Protocol (UDP) module and a transport layer Transmission Control Protocol (TCP) module as well as a session layer Real Time Transport Protocol (RTP) module, a Session Announcement Protocol (SAP) module, a Session Initiation Protocol (SIP) module and a Real Time Streaming Protocol (RTSP) module, media negotiation module, and a call control module. Portable and fixed electronic devices represented by Electronic Device 204 may include one or more additional wireless or wired interfaces in addition to the depicted IEEE 802.11 interface which may be selected from the group comprising IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000,DSL, Dial-Up, DOCSIS, Ethernet, G.hn, ISDN, MoCA, PON, and Power line communication (PLC).

Also depicted in FIG. 2 are DeLePeDa-EDs 1000 according to embodiments of the invention where within DeLePeDa-SAPs according to embodiments of the invention the Electronic Device 204 may be representative of a DeLePeDa-ED 1000. As depicted in FIG. 2 DeLePeDa-EDs 1000 according to embodiments of the invention may communicate directly to the Network 100 whilst other DeLePeDa-EDs 1000 may communicate to the Network Device 207, Access Point 206, and Electronic Device 204. Some DeLePeDa-EDs 1000 may communicate to other DeLePeDa-EDs 1000 directly. Within FIG. 2 the HPD-EDs 1000 coupled to the Network 100, Network Device 207, AP 206, and Electronic Device 204 communicate via wireless interfaces. However, within other embodiments of the invention the DeLePeDa-EDs 1000 may be coupled via wired interfaces periodically or wirelessly rather than continuously. Each DeLePeDa-ED 1000 may communicate to another electronic device, e.g. Access Point 206, Electronic Device 204 and Network Device 207, or a network, e.g. Network 100. Each DeLePeDa-EP 1000 may support one or more wireless or wired interfaces including those, for example, selected from the group comprising IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, DSL, Dial-Up, DOCSIS, Ethernet, G.hn, ISDN, MoCA, PON, and Power line communication (PLC).

Optionally, rather than wired and./or wireless communication interfaces devices may exploit other communication interfaces such as optical communication interfaces and/or satellite communications interfaces. Optical communications interfaces may support Ethernet, Gigabit Ethernet, SONET, Synchronous Digital Hierarchy (SDH) etc. An DeLePeDa-ED according to embodiments of the invention may represent a wearable device providing information to another DeLePeDa-ED, PED, or FED.

Within DeLePeDa-SAPs according to embodiments of the invention the DeLePeDa-ED may store additional information relating to the user including, for example, a user profile, user information, a computer file, and electronic content. Accordingly, the DeLePeDa-ED may employ the additional information such that within DeLePeDa-SAPs according to embodiments of the invention a DeLePeDa-SAP may encrypt some or all of the additional information into a blockchain or distributed ledger.

Within DeLePeDa-SAPs according to embodiments of the invention a DeLePeDa-ED may contain one or more sensors. For example, a sensor may include, but not be limited to, a biometric sensor, an environmental sensor, a medical sensor, a biological sensor, a chemical sensor, an ambient environment sensor, a position sensor, a motion sensor, a thermal sensor, an infrared sensor, a visible sensor, a RFID sensor, and a medical testing and diagnosis device. Accordingly, the DeLePeDa-ED may employ the data from the sensor(s) and may encrypt some or all of the additional information into a blockchain or distributed ledger. A DeLePeDa-ED according to an embodiment of the invention may be employed to provide data to a physician or other medical personnel to establish characteristics of the user either discretely, periodically, or continuously. Accordingly, a DeLePeDa-HD or DeLePaDa-SAP may track and transfer data relating to a user's physiological condition.

Decentralized Ledger Personal Data Concept

There are a number of challenges impeding the development of a personalized artificial intelligence (AI) driven health care system. Amongst, there are:
Privacy and security of client data;
A requirement for documented consent to third party use;
No way to help clients gain sovereign of their own data;
A requirement to encourage clients to share data;
Data sharing use cases;
A requirement for the control of uniform semantics and guarantees of data quality and source;
A requirement to access real world data.

Accordingly, the inventors have focused to establishing a platform, referred to within this specification as “MyPDx”, which creates an ecosystem that promotes personalized proactive health and removes barriers that stand in the way of helping them take control of their health. MyPDx thereby representing according to embodiments of the invention a DeLePaDa-SAP.

Within the following specification the inventors describe and depict two specific user cases although it would be evident that these represent only two of a plurality of use cases. The first exemplary user case, Case 1, addresses the issue of providing users with the confidence to share their personal health data to promote AI-driven personal health research. The second user case, Case 2, addresses the issue of providing users with the confidence to share their personal health data with employers and insurers through employee benefits programs where a user may be considered a customer and an insurer a payer. Both use cases address a common objective, the promotion of an AI-driven personalized healthcare that supports the ongoing emphasis shift within the healthcare profession from focusing on illness to one of focusing on wellness.

Accordingly, considering these use cases there are outlined below an overview of the main parties' “actors” involved in each use case and how each one benefits by participating in a health care ecosystem exploiting embodiments of the invention. Within the following description the abbreviation “MY” is employed to refer to Molecular You Corporation., a Canadian

Case 1—Personalized Health Research
“MY Clients” which enable privacy-preserving and secure personal health data sharing under the clients' control (e.g. users);
“MY Research Partners” which incentivize MY clients to share their data for personalized health research applications etc.;
“MY” who provides a platform supporting personal health data sharing by providing high-level privacy protection and security; and
Ethics review board which issues ethics credentials to researchers so that individuals can verify that their data will be handled properly when shared.

Case 2—Payer/Insurer
“MY clients” which enable privacy-preserving and secure personal health data sharing under the clients' control (e.g. users);
Payer/Insurer who by gaining access to data from MY clients can enable more precise insurance cost risk modelling and wellness promotion;
Employers who by gaining access to data can perform health care reviews, risk assessments, meet regulatory requirements, etc. as well as assess benefits package(s) and potentially reduce their insurance costs; and
“MY” who provides a platform supporting personal health data sharing by providing high-level privacy protection and security allowing personal health data to be employed across a number of diverse market verticals.

Accordingly, MY provides a personalized data exchange (MyPDx) which establishes a platform creating the necessary ecosystem to provide MY clients, MY research partners, payers, insurers, employers as well as a range of third party providers, service providers, enterprises, etc. It would be evident that in Case 1 and Case 2 MY Research Partners and Employers may be generalized to a Data Seekers whereas MY Clients may be generalized to Data Owners such that other use cases are clearly supported wherein a Data Seeker wishes to access one or more items of personal data, e.g. health data or biomarker, of a Data Owner. This may be a proactive wish to access, e.g. a researcher, employer, etc. or it may be a responsive wish to access, e.g. in response to a request from the data owner such as they might make to a physician, dentist, dietician, ophthalmologist, etc.

Accordingly, MyPDx provides users rather than one or more third parties with the ownership of their own health data, enabling them to choose who to share their data with, what data they share, and ultimately decide what purpose or purposes it is used for. MyPDx seeks to address and prevent the multiple instances over time of individuals' personal data being harvested, shared with, and used by third parties without informed consent and in ways that have significant potential to cause harm. Over time these events have resulted in an erosion of user trust and a reluctance to use a service that gathers their sensitive health information. This remains true even if individuals could greatly benefit from receiving a personalized health review that they can use to understand their health risks and empowers them to maintain or improve their overall health. This reluctance stems from uncertainty about how their health data services they provide data to will store and use their data over time even without consideration of data breaches, data theft etc.

Accordingly, in Europe regulators established the General Data Protection Regulation 2016/679 (commonly referred to as GDPR) which aims to give individuals control over their personal data. Business processes must provide safeguards to protect data and use the highest-possible privacy settings by default, so that the data is not available publicly without explicit informed consent and cannot be used to identify a subject. No personal data may be processed without the express unambiguous affirmation of consent from the data subject (user), who has the right to revoke this consent at any time.

However, how does a user share their information and be confident that having given consent to an enterprise that it is not subsequently sold, misused, stolen etc. Accordingly, the embodiments of the invention, for example, MyPDx, is intended to be compliant with these stricter regulations by giving users confidence and trust. This is achieved by giving individuals direct specific ownership of their data and enabling the individual to establish granular consent to data sharing of their data. Further, embodiments of the invention by providing direct granular user level control of data encourage users to share their data thereby encouraging users to be become proactive in their health, healthcare and form part of a proactive personalized health ecosystem which supports and enables a shift within the focus of healthcare from one of treating illness to one of treating wellness with a focus on the user.

Within DeLePeDa-SAPs according to embodiments of the invention the inventors exploit blockchain technology as a solution for providing private and secure data sharing. However, it would be evident that within other embodiments of the invention other distributed ledger technologies may be employed without departing from the scope of the invention. Blockchain employs confirmed and validated sets of transactions which are held in blocks chained together to make tampering difficult. Blockchain's design exploits a networked, distributed, autonomous, global operation supporting amongst a variety of features the decentralized management of privacy. A variety of blockchain platforms exist and may be employed within DeLePeDa-SAPs according to embodiments of the invention. Exemplary implementations by the inventors of embodiments of the invention exploit “Hyperledger Indy” as the blockchain platform for MyPDx. However, it would be evident that other blockchain platforms may be employed where these are either specifically designed for or modified to provide user control over their data to achieve decentralized privacy management.

Within the prior art there have been several examples of employing blockchain technology to health care records although these have primarily sought to combine so-called “cloud” computing or cloud technology in combination with blockchain to provide a security layer. Others have considered the use of blockchain technology for secure medical device to device communications or secure storage of medical insurance.

However, the embodiments of the invention shift the overall focus away from traditional centralized medical record keeping, e.g. a patient who wishes to move today from one physician to another must ask the physician to transfer their records to the other physician. Rather embodiments of the invention establish a patient (user) centric model where the patient can revoke access to their current physician and grant access to the new physician. The shift from a traditional centralized medical recordkeeping to a more patient-centric model also allows for the addition of an embedded or unembedded economic layer to compensate a patient for data contribution and use. Accordingly, within DeLePeDa-SAPs according to embodiments of the invention a user may in response to providing consent to making their health data available for research be provided with financial reward, for example, via a cryptocurrency where the reward may be determined in dependence upon the extent of the data shared and the exact data shared.

Accordingly, embodiments of the invention such as MyPDx provide a novel blockchain solution that leverages an open source blockchain framework (e.g. Hyperledger Aries/Indy) to enable privacy preserving and secure sharing of personal health data with user established granularity. The inventors initially established the MyPDx platform for the use two use cases described above. However, embodiments of the invention may support a range of other use cases within the healthcare sector. It would also be evident that embodiments of the invention may support a range of use cases in other commercial, industrial, academic, personal sectors etc. It is also evident that Case 1 differs from Case 2 in that a credential must be issued to an individual, e.g. user, allowing the researcher to verify the individual e.g., as eligible to participate in a research project and consenting to sharing their data. Revocation of the credential would thereby block access to records by that individual. Within another use case the credential may be issued by a general medical counsel which licenses physicians, for example, such that a patient can verify the physician they wish to grant access to be licensed. Again, a revocation of the physician's license and hence their credential would block access to records.

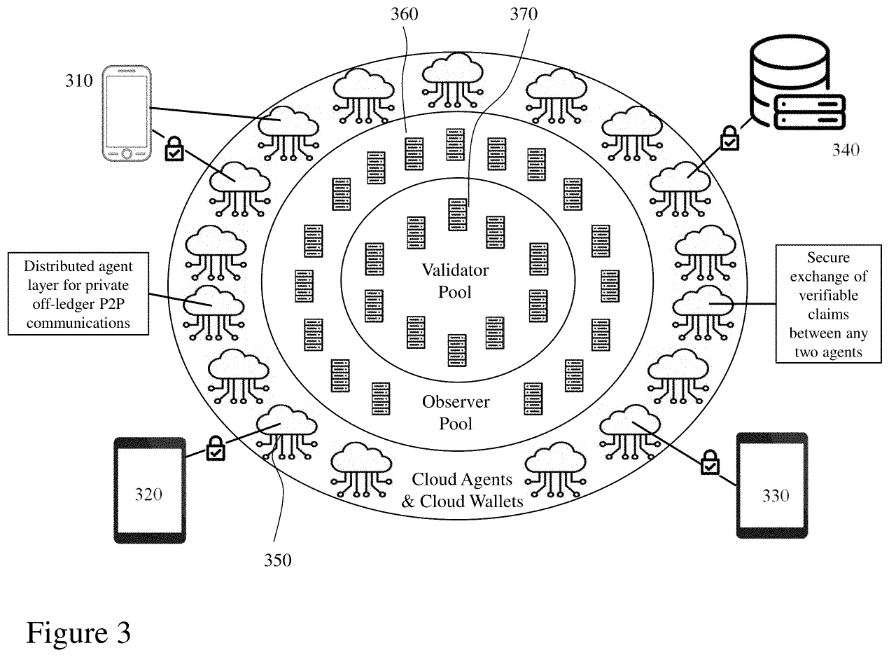

An exemplary schematic of the trust stack employed within DeLePeDa-SAPs according to embodiments of the invention is described and depicted in FIG. 3. Accordingly, users via first to third PEDs 310 to 330 or FED 340 may access cloud agents/cloud wallets 350. The plurality of cloud agents/cloud wallets 350 provide secure exchange of verifiable claims between any pair of cloud agents/cloud wallets 350. Within, conceptually, this first layer of cloud agents/cloud wallets 350 is a second layer comprising an observer pool of observers 360 monitor and manage aspects of the validation/authentication of blockchain blocks and/or a self-sovereign identity (SSI) for/by the cloud agents/cloud wallets 350 where an observer 360 may be an AI enabled device, application, or system. This second layer of observers 360 is conceptually disposed around a validator pool of validators 370 which monitor and manage aspects of the validation/authentication of blockchain blocks and/or the SSI for/by the observers 360. Accordingly, as depicted in FIG. 3 the observer pool and validator pool may be provided by an SSI network, such as that provided by the Sovrin Foundation for example, a private-sector, international non-profit organization.

Accordingly, MyPDx provides users with a novel approach to addressing the challenge of protecting the privacy and security of their health information whilst enabling them to data share for research, personal and/or business purposes. The design of the solution gives users ownership of, and control over access to their personal health data in a manner that fundamentally respects users' privacy while still allowing for and incentivizing sharing of data.

Within DeLePeDa-SAPs according to embodiments of the invention each party of the multiple parties associated with an application of a user's health data, e.g. one application may be Case 1 whilst another application may be Case 2, interacts with a blockchain wallet to perform transactions with one another. Within DeLePeDa-SAPs according to embodiments of the invention a blockchain wallet (wallet) may be a software module, and optionally an associated hardware module, for securely storing and accessing cryptographic keys (e.g. Private Keys), secrets (e.g. Link Secrets, passwords, etc.), other sensitive cryptographic key material, and other data (e.g. user Private Data) to be used by an entity (e.g. a party having permission to access the wallet). Accordingly, in use Case 1 the multiple parties are My Clients and My Research Partners whilst in Case 2 the multiple parties are My Clients with Payer/Insurer and/or Employer.

As noted above the MyPDx which represents an embodiment of a DeLePaDa-SAP supporting embodiments of the invention exploits a blockchain framework. Development of MyPDx exploiting as noted above the Hyperledger Aries/Indy blockchain framework although other blockchain and/or distributed ledger frameworks may be employed without departing from the scope of the invention provided they support or are amended to support the embodiments of the invention.

Hyperledger Aries/Indy (HL Aries/Indy) is an open source blockchain framework maintained by the Linux Foundation which provides a distributed ledger based trust layer for users across a public domain network, e.g. the Internet. As such it enables a decentralized identity management use case according to embodiments of the invention. HL is comprised of four core components:
verifiable claims;
peer-to-peer agents;
decentralized identifiers, and
a distributed ledger.

A verifiable claim within the scope of this specification is a machine-readable statement made by an entity that is cryptographically authentic and non-repudiable (i.e. a statement's author cannot successfully dispute its authorship or the validity of an associated contract). Accordingly, verifiable claims are made when an “holder” agent sends a cryptographic credential, i.e. provable digitally signed data, which is received from an “issuer” agent to another “verifier” agent across a peer-to-peer connection which the receiving verifier agent is able to cryptographically prove is authentic. Each interacting agent has a decentralized identifier, a verifiable identifier, such as an SSI that is independent from any centralized registry, identity provider, or certificate authority where the decentralized identifier is used to facilitate communication in each pairwise connection. HL Indy supports privacy-enhancing techniques, such as selective disclosure and zero-knowledge proofs (a cryptographic technique allowing an agent to prove that they know something, such as a password, without revealing what they know). A distributed ledger, a ledger that is shared across a set of nodes and synchronized between the nodes using a consensus mechanism (e.g., Practical Byzantine Fault Tolerance), is used to store Public Decentralized Identifiers (DIDs), for example of issuer agents, data schemas for credentials, credential definitions, and revocation registries (which enable cryptographic verification of claims).

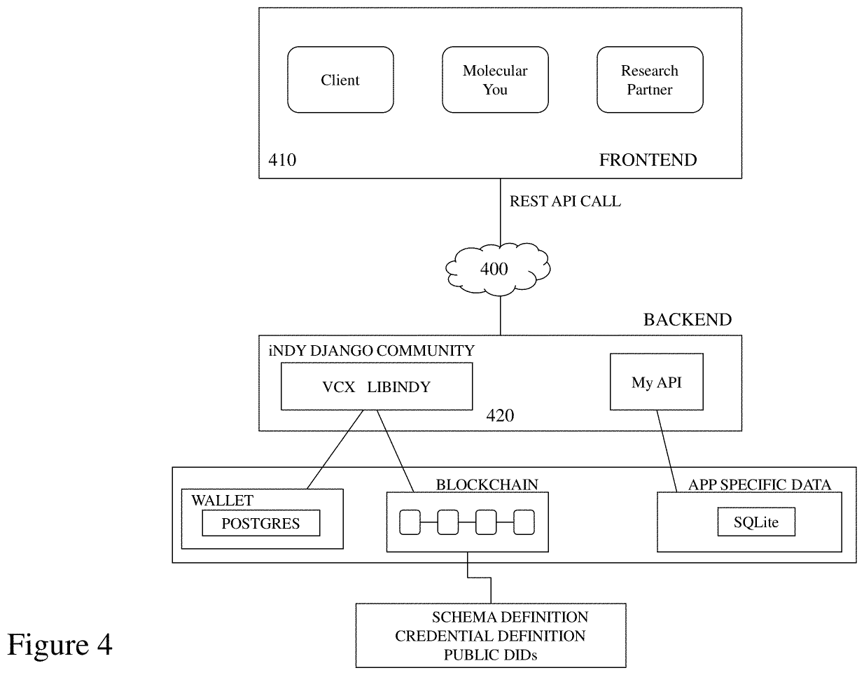

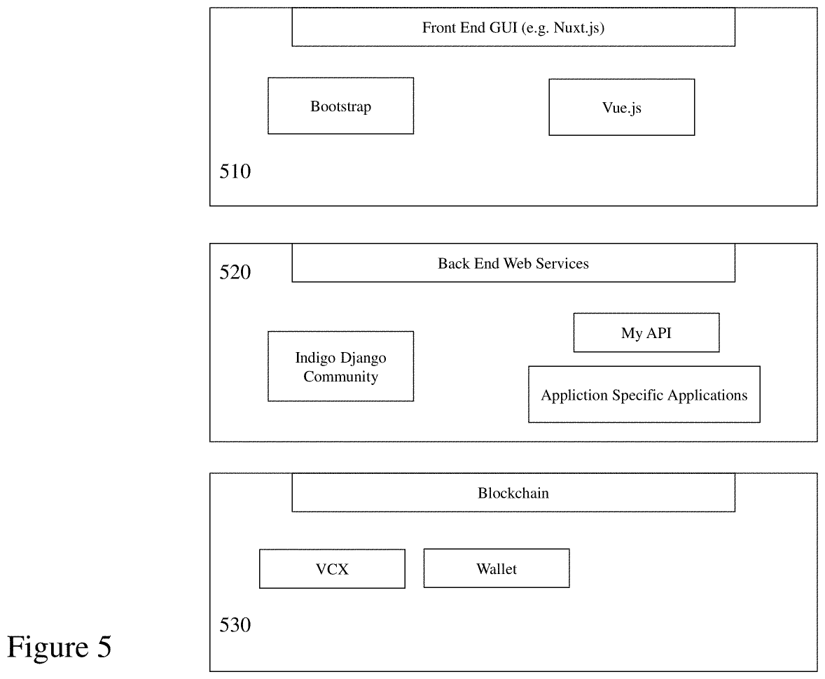

The high level architecture of FIGS. 4 and 5 representing an exemplary architecture according to an embodiment of the invention. As depicted in FIG. 4 the high-level architecture consists of a Front End 410 and a Back-End 420 which are each connected to a public network, e.g. Internet 400. The Front-End 410 includes MY which acts as a Validator/Trust Anchor and Credential Issuer to the users of the system which are the clients of Molecular You Corporation (MY), and the use case partner(s) (e.g., Research Partner who advertises their research projects for study). The front-end users communicate with the Back-End 420 using a REST API call. The Back-End 420 is designed using the Indy-Django community which is built upon LIBVCX and LIBINDY. The second component of Back-End 410 is MyAPI which has application specific data and is stored in an SQLite Database. The Indy Django community provides support for the wallet, which is created and stored in a PostgreSQL database, which is responsible for storing the credentials received by the client. Also, the Indy Django Community provides support for the Distributed Ledger i.e. the Blockchain for providing the decentralized identity. The public DIDs (Decentralized Identifiers), the schema definition and the credential definition are stored within the distributed ledger/blockchain.

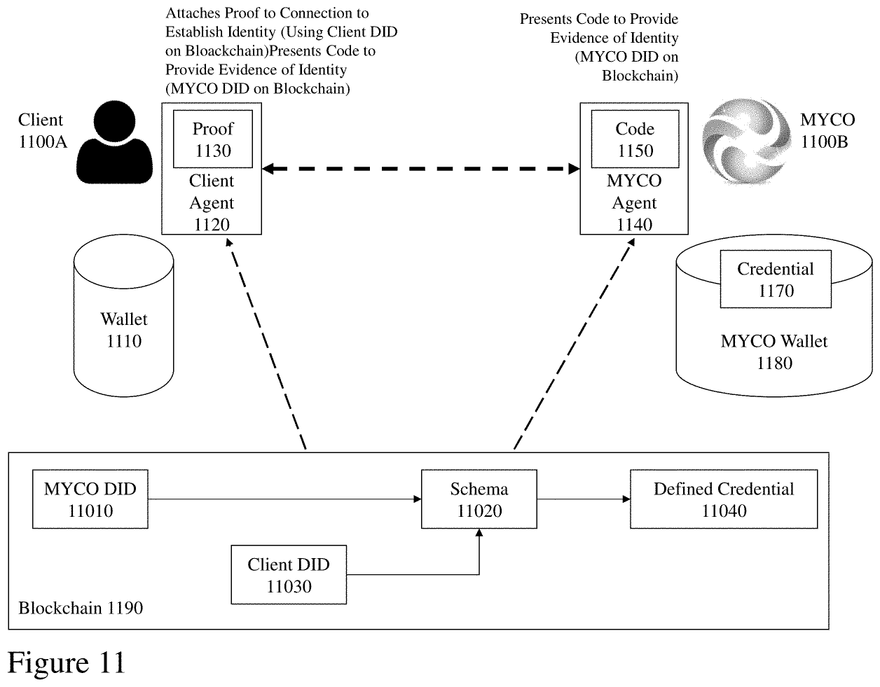

This process is depicted schematically in FIG. 11 wherein a user 1100A is initially registering for the MyPDx service and establishing data in their wallet from their stored personal data held by MYCO 1100B. Accordingly, the Client 1100A logins into a dashboard of the MyPDx service, referred to as MY Hyperledger Indy (MYHI) and requests the “Health Wallet” service. The MYHI login process identifies the client identity and establishes a session for the Client 1100A with MYCO 1100B. If the Client 1100A is establishing their health wallet (Wallet 1110) for the first time, then the MYCO Agent 1140 issues an invitation for the Client 1100A comprising Code 1150. This may, for example, be in the form of a QR code rendered to the user within the dashboard the User 1100A is currently logged in through or it may be via another communications channel such as to a user's PED based upon an identity entered by the Client 1100A during the registration process (e.g. their phone number in which case the QR code is sent to their smartphone, an email address in which case the QR code is sent to their email address etc.). It would be evident that other formats of initial data communication other than QR code may be employed to establish the invitation from the MYCO Agent 1140 to establish the Client Agent 1120. If the QR code was for example rendered within the dashboard the Client 1100A may capture an image of the QR code wherein the Client Agent 1120 is established. The Client 1100A then accepts the invitation. This thereby creates a Credential 1170 within the MYCO Wallet 1180 which points to Defined Credential 11040 upon the Blockchain 1190. Accordingly, using the connection already created between the Client Agent 1120 and the MYCO Wallet 1180 via MYCO Agent 1140 the Client 1120 can request personal data from the report (i.e. records held by MYCO) which are transferred to their Wallet 1110.

The Code 1150 presented to the Client 1110A as proof of identity of MYCO 1100B points to the MYCO DID 11010 on the Blockchain 1190. Similarly, the Proof 1130 presented to MYCO 1100B by the Client 1110A as proof of identity of Client 1100A points to the Client DID 11030 on the Blockchain 1190. The Defined Credential 11040 pointed to by Credential 1170 within MYCO Wallet 1180 is a unique credential established by applying a Schema 11020 to the MYCO DID 11010 and Client DID 11030. As will become evident with respect to DeLePeDa-SAPs according to embodiments of the invention the Client DID 11030 and/or the MYCO DID 11010 may be established uniquely for each request from Client 1100A to MYCO 1100B such that every request for personal data is associated with a unique Defined Credential 11040. Further, as will become evident with respect to DeLePeDa-SAPs according to embodiments of the invention this Defined Credential 11040 may provide part of an audit trail that Client 1100A did indeed request personal data from MYCO 1100B. As the personal data is transferred from MYCO Wallet 1180 to Wallet 1110 without being stored in the Blockchain 1190 the resulting audit trail does not contain any personal data of the Client 1100A. The exemplary process depicted in FIG. 11 may be viewed in reverse where MYCO 1100B wishes to obtain personal data from Client 1100A.

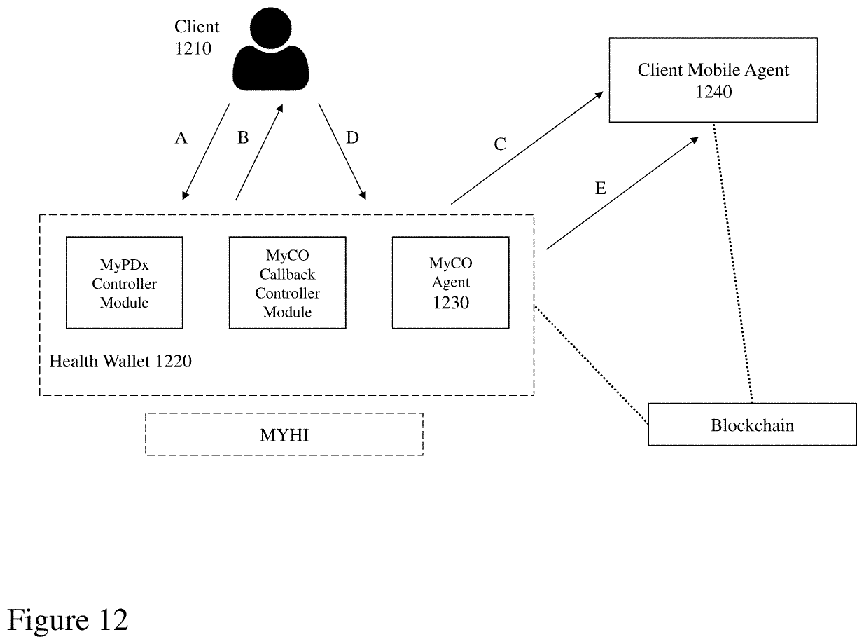

This exemplary process is also depicted in FIG. 12 showing the steps A through E respectively wherein:
Step A: Client logins into MYHI dashboard and requests the Health Wallet 1220 via the “Health Wallet” service and the MYHI login identifies the client's identity;
Step B: For a client getting the health wallet for the first time, the MyCO Agent 1230 issues an invitation for Client 1210 in the form of QR code (for example);
Step C: Client 1210 scans the invitation in the Client Mobile Agent 1240 and accepts invitation;
Step D: Using the connection already created between the Client Mobile Agent 1240 and Health Wallet 1220 the Client 1210 can request credentials from their report; and
Step E: MyCO issues Health Credentials to Client Mobile Agent 1240 and therein to their personal wallet.

It would be evident within other embodiments of the invention that rather than requesting the “Health Wallet” the Client 1210 may request “Financial Wallet” relating to financial data, “Personal Wallet” relating to personal data, “Academic Wallet” relating to academic data, etc. without departing from the scope of the invention.

As depicted in FIG. 5 the Front End 510 provides the user with a Graphical User Interface (GUI) as a web page(s) which within a DeLePeDa-SAP according to embodiments of the invention may exploit, for example, Nuxt.js which is built on the top of vue.js. The inventors adopted Vue framework for the development of web-pages is that supports an incrementally adaptable architecture plus provides various optional tools for development and facilitates integration with existing applications. The GUI web pages for initial development implementations were designed using Bootstrap for its front-end CSS library allowing the inventors to rapidly prototype responsive mobile and web applications.

The Front End 510 of the web based application communicates via the Internet with Back End Web Services 529 which within the prototypes developed by the inventors exploited the Indy-Django Community library which is a python library providing a cloud-based framework for developing Hyperledger Indy Agent applications. Indy Django provides the support for LIBVCX and LIBINDY, the Hyperledger Indy libraries. LIBINDY is implemented using a foreign function interface (FFI) to a native library written in Rust. LIBVCX is a c-callable library built on top of LIBINDY that provides a high-level credential exchange protocol. LIBVCX helps in creation of agent applications and provides better agent-2-agent interoperability for Hyperledger Indy infrastructure.

The application related APIs are contained in MyAPI, which encompasses the project data within it. The users entering the system such as validators/trust anchors, credential issuers, research partners or the clients are defined and created as a part of MyAPI. The Indy Django community provides support for wallet services as well wherein within DeLePeDa-SAPs according to embodiments of the invention the wallet holds the credentials for the client received from a trust anchor in encrypted form. The wallet for prototypes of MyPDx being implemented in PostgreSQL.

As noted above in respect of the two representative user cases, Case 1, and Case 2, the MyPDx initial prototype implementations have four main interacting parties although it would be evident to one skilled in the art that the number of parties within an application of embodiments of the invention may vary from two upwards. Accordingly, the first step in an application flow occurs when a MY client requests cryptographic credentials for each of their biomarkers. MY then issues these credentials to the personal health wallet of each individual data owner (MY client). As the representative user cases Case 1 and Case 2 relate to health based applications the requests from a client relate to health information of the user, namely biomarkers. However, within other usage cases the data the user wishes to share and accordingly requests cryptographic credentials for may be other data including, but not limited to, personal data.

Considering Case 1 then according to an embodiment of the invention the research makes an application to the Ethics Review Board (ERB) for an ethics certificate to conduct their research and, once approved, an ethics certificate is sent in the form of a cryptographic credential to the wallet of the applicant/researcher. Once the researcher has ethics approval then they are able to use the MyPDx platform to advertise their research projects to data owners (i.e. users/patients).

When a data owner notices a research project in which they would like to participate, this initiates a “handshake” process using a Hyperledger Indy-based blockchain network in which the data owner verifies that the research project has the necessary ethics approval, and the researcher verifies that the data owner meets their study criteria and obtains consent to sharing their data. The process completes when the data owner shares their specific health data (e.g., biomarkers) needed for the study. Optionally, the researcher or a sponsor of the research may send the data owner a reward for their participation in the study, which may be in the cryptographic credential (e.g. a cryptographic currency reward). Typically, the reward would adhere to the principles of the Global Alliance for Genomics and Health (GA4GH) which allows for researchers to recognize data owners for sharing their data in a manner that is meaningful and appropriate.

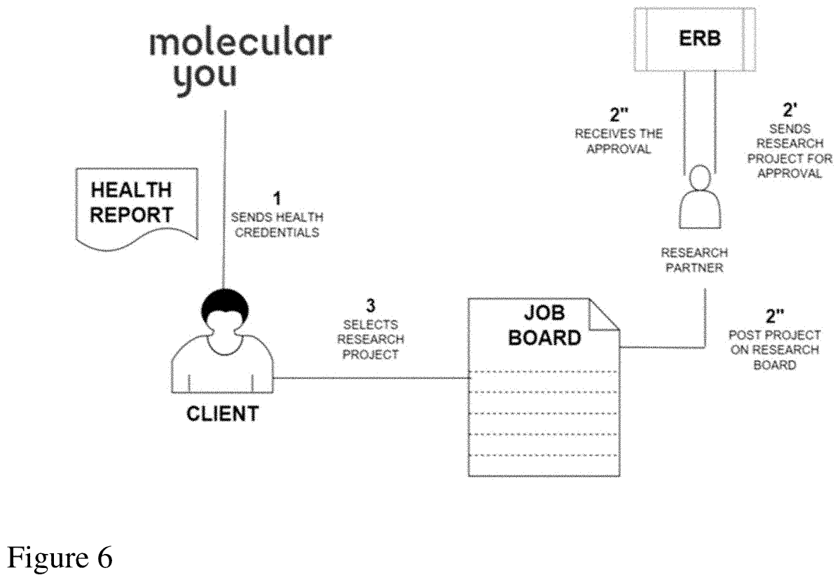

FIG. 6 provides a visual overview of this Case 1 (Research Use Case) application flow which takes place between data owners (i.e., MY clients) and researchers on MyPDx. A key feature of the entire process is that the identity of data owners is never revealed to researchers, no personal health information is ever recorded or stored on the blockchain, and data owners remain in control of their personal health information at all times, revealing only as much information as they feel comfortable with given their assessment of the risk-benefits of the transaction. The entire transaction takes place via a wallet which only exists for that specific relationship between data owner and researcher with only them having cryptographic credentials to place data into and acquire data from the wallet, respectively.

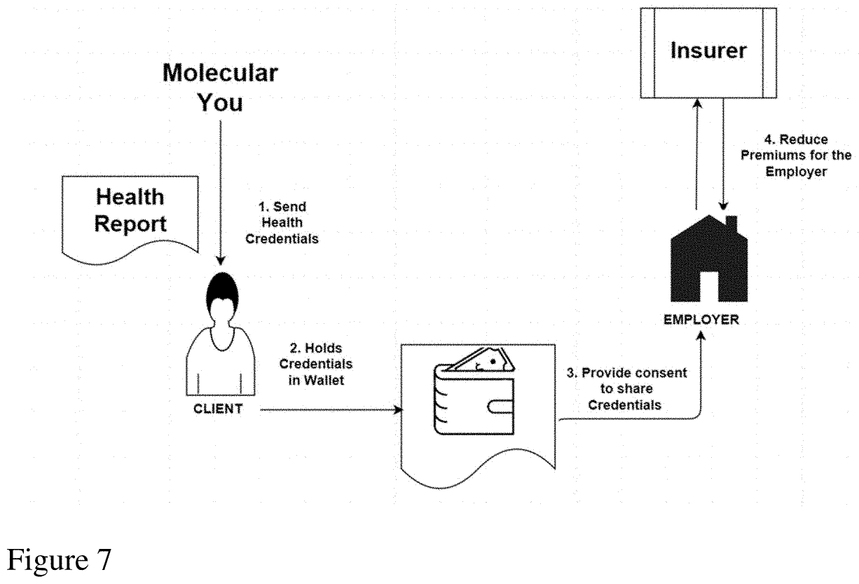

Now considering the second exemplary user case, Case 2, then the exemplary application flow is depicted in FIG. 7. Accordingly, this use case of an embodiment of the invention in MyPDx again has four main parties. However, this application flow enables:
MY to issue health credentials to employees certifying their current level of wellness;
Employees to share their health credentials as secure and privacy preserving zero knowledge proof verifiable claims with employers/insurers;
Insurers to instantly see on aggregate the wellness trajectory of an employer's employee population and adjust insurance premiums accordingly without the employer or the insurer needing to know or track any personal health information about individual employees.

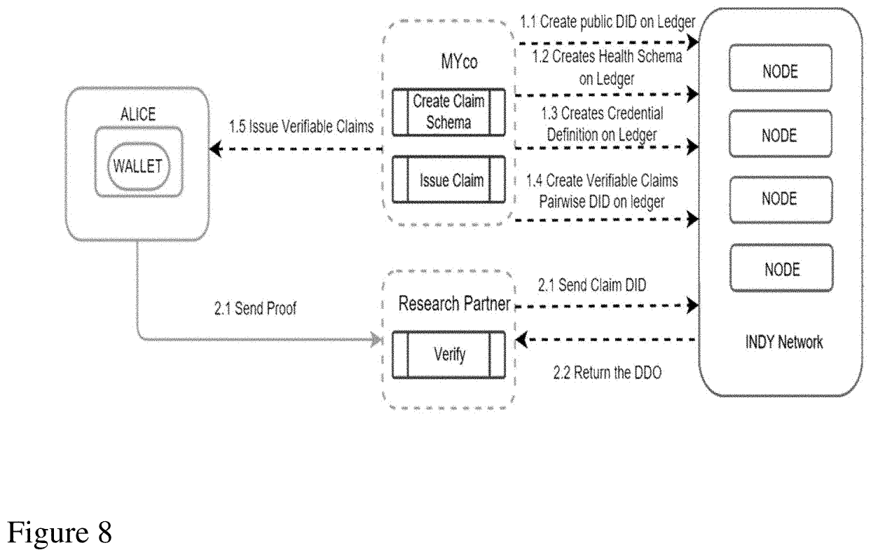

Accordingly, the employer and/or insurer may provide to an employee a reward for sharing information discretely or in combination with ongoing incentivizes based upon follow up actions, data sharing etc. wherein an employee may be incentivized to follow a user specific healthcare plan and adopt healthier behaviours. It would be evident that the initial step of MY issuing a health credential may be performed using a process flow such as described and depicted with respect to FIG. 8 and Case 1 where the biomarker(s) shared are processed to determine a result which is used to determine the current level of wellness of the employee.

Accordingly, such a use case provides those participating in the secure ecosystem with benefits for each party. For employees they may receive regular information to help them stay healthy and rewards for doing so without fearing that their personal health information will be exposed. For employers they can promote a wellness oriented work culture and receive reductions in their insurance premiums. For insurers', the cost of insuring for sickness goes down as overall wellness increases and they have a secure, privacy preserving data source for use in precision tuning of risk models.

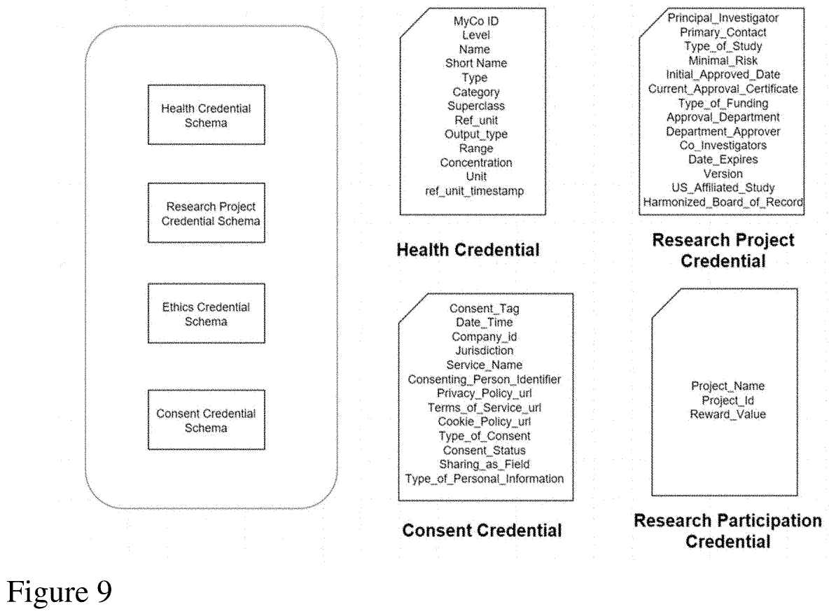

It is important to note that within the embodiments of the invention such as the prototype MyPDx established by the users that no personal health data is recorded on the blockchain. Instead, an embodiment of the invention such as MyPDx only stores Public Decentralized Identifiers (Public DIDs), Credential Schemas, and Credential Definitions on the blockchain. Accordingly, referring to FIG. 9 there is depicted an overview of the Credential Schemas and Credential Definitions for Case 1, the Research Use Case. In this example the user is named “Alice.” As depicted the flows do not include those relating to the initial request from Alice to establish their wallet, those related to the researcher credential generation etc.

It would be evident that an embodiment of the invention operates as a distributed system with components operated by independently controlled and federated entities (validator nodes). Components may be maintained on-premises or in the cloud (e.g., an Infrastructure-as-a-Service (IaaS) model). The front-end and back-end components employ a client-server deployment model, with the front end (e.g., devices, web apps) making calls to a backend server, which then communicates with the blockchain. Responsibility and location for operation of components may within DeLePeDa-SAPs according to embodiments of the invention be as follows:
Solution Frontend—An application provided by MY but operated on a client's endpoint device or a partner's endpoint device (e.g., PED or FED directly or via a web based application through a web browser). MY also provides support for databases used for the storage of each client's DID pairs and credentials, and the database used for storage of app-specific data along with client's credentials. Each of these databases may be maintained in the cloud.
Solution Backend—Nodes in the validator pool, e.g., Trust Anchors and/or Credential Issuers, which are controlled and operated by independent ecosystem partners, each of which may, for example, be a separate legal entity. Maintaining and operating these nodes is the responsibility of the individual partners. They may operate the nodes on-premises or in the cloud.

Accordingly, the inventors inventive concept exploits Self Sovereign Identity (SSI) so that the individual has custody and control of their own data and can manage who has access to their data and for what purpose. The blockchain is employed to provide a platform for managing and distributing public identities and document definitions that are used to facilitate off-ledger transactions. Accordingly, the individual's personal data is not written to the blockchain thereby providing users with increased security and confidence in the management of their personal data. Further, within DeLePeDa-SAPs according to embodiments of the invention the actual transactions between parties such as issuing and validating are not written to the blockchain ledger. Accordingly, agents within DeLePeDa-SAPs according to embodiments of the invention provide the facility for applications to connect and exchange data in a secure and privacy-preserving manner. Accordingly, the inventor's methodology exploits and implements SSI in applications that provide to users inherent “Privacy by Design.” Accordingly, the embodiments of the invention provide a framework exploiting decentralized identity and verifiable credentials via a distributed ledger absent the storage of personal data within the distributed ledger.

Embodiments of the invention exploit technological developments which enable new forms of human engagement and coordination to transform healthcare data management and sharing. Accordingly, embodiments of the invention exploit a blockchain to create a new technological infrastructure for the establishment of decentralized cooperation with no central coordinating, monitoring, or ruling authority. Accordingly, the inventors inventive concept by providing personal data management in the hands of the user and providing for its sharing in a secure manner without storage of the personal data in the publicly accessible distributed ledger (even if it is encrypted) will enable new forms of value creation and distribution with or without the issuance and transfer of rewards. The MyPDx solution anticipates new methods of value creation and distribution, both internally within the ecosystem, and externally between the ecosystem and third parties. These come along with a new economic model relying upon the formation of digital rewards, coupled to the services the MyPDx solution provides.

As discussed above embodiments of the invention are built on the foundation that each customer has a self-sovereign identity (SSI). Within DeLePeDa-SAPs according to embodiments of the invention this SSI may be an inalienable digital health identity based on the user's biomarkers. It is expected that early adopters of the MyPDx platform and DeLaPeDa-SAPs according to embodiments of the invention will be health conscious individuals before enterprises such as employers, insurers, regulators etc. require personal data sharing for specific objectives. DeLePeDa-SAPs according to embodiments of the invention securely exchange data, for example with researchers, employers, and payers, in a privacy-preserving manner. A variety of factors are expected to drive the adoption of the DeLePeDa-SAPs according to embodiments of the invention. These factors are set against a health context wherein the current health care delivery model is inverted from one of business-to-consumer (B2C) to consumer-to-business (C2B). This means consumers are pulling services, rather than being pushed services. The future of healthcare delivery is now expected to leverage and employ innovative technologies and personalized programs with a focus on data interoperability, privacy, security, and ownership. The new context has emerged from an ageing population with disposable income, use of mobile and online healthcare tools, a strained healthcare system, more techno-literate end users with a core tenet of privacy, and rising data security risks.

Factors impacting customer/end-user adoption of embodiments of the invention such as MyPDx may include, but not be limited, to:
Concerns about data privacy and security;
Concerns about control and consent;
Path to a join a marketplace where causes and research to which the end user feels attached and where their data could provide value i.e. cancer research;
End-user receives a reward in compensation for consenting to share data;
Potential for flexible insurance policies through sharing of data with the reduction of price based on agreed-to metrics;
Personalized insurance policies;
Social Health networks for information, motivation, and support;
Self-directed/self-diagnostics health care;
Personal data exchange with physician and health coach; and
Personalized health improvement plans.

Factors impacting payer/researcher/employer etc. adoption of embodiments of the invention such as MyPDx may include, but not be limited to:
Consented access to data;
Compliance to new privacy regulations and consumer expectations of data control;
Pay only for the data consumed;
Improved and easy-to-use platform to share data between parties in a secure manner;
Marketplace reduces the friction between data buyers and sellers which lowers the cost of acquiring the data;
Provision of high quality authenticated/verifiable data;
Offer better rates and better risk management models for insurance policies and employee benefit platforms;
Participation-based programs with consented data;
Outcome-based programs based on real-time health data;
Access to and better leveraging big data and smart dashboard;
Mobile health technologies, wearables, and other digital programs;
Better data analytics of health, content, and customized/channel optimization; and
Benefits and formulary management for insurance and employee benefit programs.

Within DeLePeDa-SAPs according to embodiments of the invention a consortium partner may operate a single validator node to make this a truly decentralized, though permissioned, platform, which will be, at the same time, capable of interacting with a larger public network (e.g., an SSI network). Consortium partners and those operating validator nodes or as trust anchors may be private for-profit entities, private not-for-profit entities, or public institutions for example. Within DeLePeDa-SAPs according to embodiments of the invention there may be a subscription fee for data purchasers and a transaction fee paid from the data purchaser to the end-user. Within DeLePeDa-SAPs according to embodiments of the invention an end user, once on-boarded as a MY client, will have access to the MyPDx wallet to store their healthcare credentials. This may be free of charge, or a cost may be charged due to the security benefits that the embodiments of the invention provide relative to other methodologies.

, This fee may, for example, be a monthly subscription to sell and store health data. This transaction fee may be market-based rather than linked to a token or it may be linked to a token. For example, a fee per medical record may vary from a low figure, e.g. $0.10, to a high value, e.g. $1,000, according to the completeness of the records.

Within DeLePeDa-SAPs according to embodiments of the invention a breakdown of the payments will be delivered through the platform (e.g., via smart contract). A portion may go to the data holder (i.e., end user−% of total payment), credential proofer (% paid to an issuer for each time its credential is used to prove a verifiable claim), network maintenance (%, representing ongoing value contribution or stake in the network used for network maintenance), and to the consortium partners (i.e., validator nodes−% divided among all partners according to their initial stake and ongoing stake in the network).

It would be evident that the consent-based data marketplace for healthcare data has the potential to impact all markets. Data is becoming the most important tool for the growth of industries. Consumer data, family data, health data, and geospatial data are all examples of personal data that can help any business make decisions and transform. The privacy expectations of data are cross-cutting and apply to all data that can be monetized, but this must be done with the consumer in control by exploiting DeLePeDa-SAPs according to embodiments of the invention.

Beneficially, embodiments of the invention such as MyPDx provide a permissioned network which works with public networks and other specific organization networks. Whilst embodiments of the invention may exploit a speculative token; e.g. bitcoin etc., this is not central to the operation of DeLePeDa-SAPs according to embodiments of the invention and these may therefore leverage the frictionless benefits of tokenization of flat currencies at reduced costs and increased speeds. In opposition to developing a marketplace for health data and then improving to implement sensitive healthcare data security, DeLePeDa-SAPs such as MyPDx are compliant with requirements such as Canada's Personal Information Protection and Electronic Documents Act (PIPEDA), the United States of America's Health Insurance Portability and Accountability Act (HIPAA), Europe's General Data Protection Regulation (GDPR) and the United States Food and Drug Administration Regulation 21 CFR Part 11 (Electronic Records and Electronic Signatures).

DeLePeDa-SAPs according to embodiments of the invention are expected to open and facilitate new methods of value creation and distribution, both internally within the ecosystem, and externally between the ecosystem and third parties. By exploiting an open source model to the software implementing DeLePeDa-SAPs according to embodiments of the invention the inventors are establishing an ecosystem easily accessible to new innovators and researchers and interoperable with wider national and international networks. DeLePeDa-SAPs according to embodiments of the invention may establish non-commercial and commercial models based on trust in sharing data allowing DeLePeDa-SAPs according to embodiments of the invention to be leveraged to build new shared assets. It is anticipated that DeLePeDa-SAPs according to embodiments of the invention will encourage researchers, clinicians, and patients to interact directly as well as with innovators in ways never before possible. Business opportunities and collaborations will be taken to a new level where trust is at the foundation of every transaction.

DeLePeDa-SAPs according to embodiments of the invention have been established by the inventors based upon four underlying concepts and their associated underpinnings thereby impacting the solution design and differentiating this solution from prior art blockchain solutions. These concepts being:
Privacy by design;
Global Alliance for Genetic Health (GA4GH);
Self-sovereign identity; and
Respect.

Privacy by design (PbD) is driven not by compliance with regulatory frameworks but rather by an organization's default mode of operation and takes within its view its IT systems, accountable business practices, networked infrastructure. Accordingly, DeLePeDa-SAPs according to embodiments of the invention are based upon a PbD framework consists of seven foundational principles:
proactive not reactive: preventative not remedial, which speaks to the fundamental idea building privacy into systems at the point of design, as opposed to adding them later;
privacy as the default setting in IT systems;
privacy embedded into design, which encompasses the notion that privacy should never be bolted on to an existing solution;
full functionality: positive-sum, not zero-sum;
end-to-end security: full lifecycle protection; visibility and transparency: keep it open, which speaks to the requirement to make data subjects fully aware of how their personal data being collected and used, and for what purpose(s); and

respect for user privacy.
Global Alliance for Genetic Health (GA4GH)'s Framework for Responsible Sharing of Genomic and Health-Related Data addresses participation within what is commonly referred to as omic research, i.e. those branches of science and/or disciplines in biology whose names end in the suffix -omics. Accordingly, designing DeLePeDa-SAPs according to embodiments of the invention to be compliant with the GA4GH framework emphasizes:
transparency, which entails developing clearly defined and accessible information on the purposes, processes, procedures, and governance frameworks for data sharing; providing clear information on the purpose, collection, use and exchange of genomic and health-related data; and implementing procedures for fairly determining requests for data access and/or exchange;
accountability, which includes tracking the chain of data access and/or exchange to its source;
data quality and security, encompassing storing and process data collected, used, and transferred in a way that is accurate, verifiable, unbiased, proportionate, and current, so as to enhance their interoperability and replicability and also preserve their long-term searchability and integrity;
compliance with applicable privacy and data protection regulations at every stage of data sharing, with assurances to users that confidentiality and privacy are appropriately protected when data are collected, stored, processed, and exchanged;
risk-benefit analysis of the harms and benefits of data sharing on and with individuals, families, and communities;
recognition and attribution for data sharing that are meaningful and appropriate to the medium or discipline concerned, and which provide due credit and acknowledgement of all who contributed to the results;
sustainability of the data generated for future use, through both archiving and using appropriate identification and retrieval systems, and through critical appraisal of the mechanisms and systems used for sharing genomic and health-related data;
education and training so as to advance data sharing and data management and to constantly improve data quality and integrity; and
accessibility and dissemination, which includes collaborative partnerships and data sharing that can generate maximum benefit, along with the harmonization of deposit, management and access procedures and use as a means to promote accessibility.

Self-Sovereign Identity (SSI), a variant on decentralized digital identity, within DeLePeDa-SAPs according to embodiments of the invention leverages the affordances of blockchain technology to increase user control over their identity in the digital world. SSI implies that an individuals' identities and the data associated with them are neither bestowed, revocable nor owned by any authority save for the individual themselves. Accordingly, DeLePeDa-SAPs according to embodiments of the invention establish SSI as a layer within an Open Systems Interconnection (OSI) model stack allowing DeLePeDa-SAPs according to embodiments of the invention to satisfy three basic requirements:
Security—the identity information must be protected from unintentional disclosure;
Control—the identity owner must be in control of who can see and access their data and for what purposes; and
Portability—the user must be able to use their identity data wherever they want and not be tied into a single provider.

Accordingly, DeLePeDa-SAPs according to embodiments of the invention employ SSI to build upon PbD and GA4GH models so that the user becomes controller of their health related data, for example, rather than this control being held by data stewards, research ethics boards, and researchers for example. Accordingly, the tenets of SSI embedded within DeLePeDa-SAPs according to embodiments of the invention are:
every individual human being is the original source of their own identity;
identity is not an administrative mechanism for others to control;
each individual is the root of their own identity, and central to its administration.

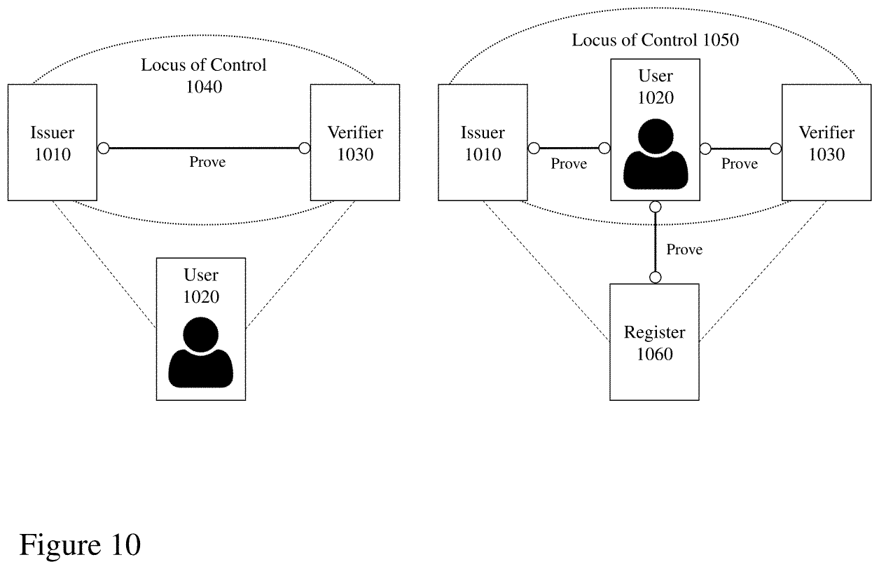

Referring to FIG. 10 there are depicted the prior art locus of control of data and the locus of control provided by SSI within DeLePeDa-SAPs according to embodiments of the invention.

Respect addresses privacy issues quite differently from other bio-centric ethical frames as the question is changed from what ought to be done (e.g., whether to allow secondary research use of individuals' health data) to what ought to be respected (e.g. the user to whom the data, e.g. medical data relates) or improved (e.g., the infosphere, which is the environment constituted by the totality of information entities—including all agents—processes, their properties, and mutual relations).

DeLePeDa-SAPs according to embodiments of the invention operate as a distributed system comprised of networked endpoint devices, APIs, database servers, code libraries, etc. In terms of the core open source HL Aries/Indy code and HL Ursa cryptography upon which the privacy-preserving and secure data exchange features of this solution depend, the Linux Foundation, which maintains the HL Aries/Indy codebase, undertakes periodic security audits. However, the overall security of the MyPDx solution depends on assessing and assuring the security of all its interacting components and is only as good as its weakest link. However, fundamentally DeLePeDa-SAPs according to embodiments of the invention do not store personal data within the blockchain. Whilst most discussions concerning the security of blockchain are focused to the difficulty of changing a transaction within blockchain (e.g. not only the initial transaction blockchain block but all subsequent blockchain blocks would require manipulation) the issue of data privacy is different in that unauthorized decrypting the content of even a single blockchain block that contains personal data is a breach. Accordingly, DeLePeDa-SAPs according to embodiments of the invention avoid this issue by using blockchain solely to negotiate/authorise transfers of data whilst the actual data transfer is performed through a cryptographically encrypted wallet which is of itself not public.

DeLePeDa-SAPs according to embodiments of the invention exploit validator nodes which operate according to an acceptable service level of issues such as 24/7 connectivity, response time, minimal downtime, etc.

DeLePeDa-SAPs according to embodiments of the invention such as MyPDx operate as a permissioned blockchain; that is, authorization will be required to perform activities using the solution. This implies the need for an identity for the entity involved, to which authorization is granted. There will be both organization identities and also identities for individuals (e.g. MY clients). Accordingly, within DeLePeDa-SAPs according to embodiments of the invention an organization may be required to register as a MY partner thereby agreeing to the terms and conditions, including those pertaining to maintenance of privacy and security, of participating in the ecosystem. Once registered, partner organizations will create their own MyPDx wallet(s), which will generate a fixed Public DID that the partner will use to issue credentials. The Public DIDs of all credential issuers (i.e., partner organizations) will be stored on the Blockchain.

All individuals participating in the network may be required to register as a MY client. A registered MY client can participate in the MyPDx network or not, this is entirely their choice. If they choose to participate, MY clients will create a MyPDx wallet after agreeing to the terms and conditions of participating in the ecosystem. Privacy-preserving and secure integration of client wallets with MY's operational systems will be undertaken as part of DeLePeDa-SAPs according to embodiments of the invention.

DeLePeDa-SAPs according to embodiments of the invention exploit a storage architecture which may be viewed as having three primary components:
data storage on the blockchain;
data associated with client wallets; and
application specific data.

With respect to DeLePeDa-SAPs according to embodiments of the invention and the data stored on the blockchain (e.g., Public DIDs of issuers, Credential Schema, Credential Definitions and, a credential revocation registry) these may be stored on each of the nodes operating the blockchain solution. Accordingly, details of data storage on Indy nodes are discussed here but other implementations may be employed within DeLePeDa-SAPs according to embodiments of the invention without departing from the scope of the invention. Blockchain nodes are operated by participating partners designated as “Trust Anchors” (i.e., partners who are trusted to maintain the network). Participating partners may choose to maintain their nodes in-house or in the cloud (by a provider of their choosing provided the service meets with the ecosystems' terms and conditions).

Data associated with client wallets (i.e., pair-wise DIDs, credentials) within DeLePeDa-SAPs according to embodiments of the invention is stored off-blockchain, e.g. within a PostgreSQL database in the cloud for example. Each client will have their own database logically segregated from the database of all other clients. Optionally, a client within DeLePeDa-SAPs according to embodiments of the invention may be able to connect their wallet application to a backend storage repository of their choosing, in keeping with the principle of self-sovereignty, rather than one defined by the DeLePeDa-SAP for example.

Application specific data (e.g., research posts) may be stored within a network accessible database, for example an SQLite database, which will operate in the cloud. DeLePeDa-SAPs according to embodiments of the invention may be agnostic as to cloud service provider provided that they offer compliant services, e.g. PIPEDA/HIPAA compliant services.

DeLePeDa-SAPs according to embodiments of the invention may implement a Key and Data Backup Strategy which may, for example, be a decentralized key management system (DKMS) allowing for users to access a password recovery/backup mechanism.

DeLePeDa-SAPs according to embodiments of the invention may backup credential stores, e.g. backing up cloud-based databases, which may be established in compliance with one or more industry standard such as ISO 27000 for example.

Data recorded on the blockchain by DeLePeDa-SAPs according to embodiments of the invention may be distributed out to network nodes (e.g., credential schemas, definitions, public DIDs), with each node serving as a backup copy to the others.

DeLePeDa-SAPs according to embodiments of the invention fundamentally shift Data Stewardship Ownership and Control, i.e. the sovereignty of data, to each individual client. Accordingly, clients own and control credentials issued to them by DeLePeDa-SAPs according to embodiments of the invention and other credential issuers. Each credential issuer will define, control, and maintain the schema and credential definitions it issues unless the credential schema is used by more than one issuer e.g., consent credential. In that case, the schema and associated credential definitions may be defined in consultation with ecosystem partners and reference relevant standards and will be controlled and maintained by an entity, e.g. MY, on behalf of the ecosystem.

DeLePeDa-SAPs according to embodiments of the invention may also provide for Data Source Tracking as defined by the functionality of the blockchain framework. For example, a blockchain fabric may cryptographically authenticate the source of all data. Thus, users can make factual claims based on the credentials they hold that can be cryptographically proven as authentic (i.e., issued by a trusted source and having integrity). A cryptographic proof may be, for example a JavaScript Object Notification (JSON) document that contains some revealed or zero-knowledge proof attributes, plus signatures that prove that the attributes were attested to by the issuers of credentials. In some instances, the attributes come directly from a proof (e.g., a biomarker name); sometimes it is communicated as the mathematical demonstration of a predicate (the fact that a biomarker has a certain value). Verification consists of checking the math used by the prover and of checking the signatures to see that they were created by the appropriate keys.

In some instances, a DeLePeDa-SAP according to an embodiment of the invention may be associated with an enterprise which also provides health data. For example, MY may provide not only the MyPDx application (a DeLePeDa-SAP according to an embodiment of the invention) but also provide data to clients which is then subsequently transferred using the exemplary processes described and depicted above. In such instances the transfer of personal data from MY to the client may exploit the processes and flows described and depicted with respect to MyPDx.

Within DeLePeDa-SAPs according to embodiments of the invention data may be exchanged using Peer-to-Peer Data Exchange. A digital verifiable credential is a piece of information that is cryptographically trustworthy. In order to enhance the privacy and security of the data being sent, the blockchain framework employed, e.g. HL Indy, may have the capability to provide privacy using Zero-Knowledge Proofs and Selective Disclosure. With such a blockchain framework and a built-in zero-knowledge proof, it is possible to verify any claim without giving out any personally identifiable information (PII). Public verification, in general, includes all PII data, making the identity information constantly vulnerable. DeLePeDa-SAPs according to embodiments of the invention exploit data minimization, i.e. the collection of personal information to that which is directly relevant and necessary to accomplish a specified purpose.

DeLePeDa-SAPs according to embodiments of the invention may also address another client issue with respect of their personal data, i.e. how do you prove what happened to it. DeLePeDa-SAPs according to embodiments of the invention allow for the implementation of audit trails which are designed, in addition to the other underlying tenets of the DeLePeDa-SAP design and implementation, to resolve several barriers to trust that often prevent individuals from sharing their personal health data or any other personal data.

A common first barrier to sharing occurs because individuals are reluctant to use a service that gathers sensitive health information about them, even if they benefit from receiving a personalized health review that they can use to understand their health risks and which, by delivery of a personalized action plan, empowers them to improve their overall wellness. Individuals' reluctance stems from uncertainty about how the service will store and possibly use their health data at a future time. Given recent revelations about how major social networks and other platforms use individuals' sensitive personal data, users rightly fear that their data will be shared with third parties without their consent, even when the service provider's privacy policy clear states that the company will not do this. Individuals' trust has been broken and, as a result, they are increasingly unwilling to share their data even for legitimate and personally or socially beneficial purposes, such as ethically certificated health research. DeLePeDa-SAPs according to embodiments of the invention by making individual users the owners of their health data and enabling the user to define granular levels of consent to data sharing give users greater confidence and increase their trust in the platform and the data sharing process. SSI means the user is in control and knows they are.

Users also usually are very concerned that their identities will be linked to information about their health, as already discussed. In particular, individuals whose biomarkers indicate that they have, or are at risk for, certain types of diseases may fear that they will be discriminated against by insurers or employers, or that they will be socially ostracized. These risks make it extremely important to ensure that it is not possible to connect users' personal health data with their real-world identities. The solution protects the real-world identities associated with specific biomarkers through the use of decentralized identifiers (DIDs). With one identity for everything it is easy to correlate an individual with their health data. In contrast DeLePeDa-SAPs according to embodiments of the invention employ a different identity for every relationship and transaction, i.e. DeLePeDa-SAPs according to embodiments of the invention use separate DIDs for each pair-wise interaction. Further, these DIDs are controlled by the individual and stored in their wallet rather than in the blockchain.

Another barrier to trust is that requests for access to information often demand more information than necessary from users. For example, a study may request access to an individual's entire genetic sequence or a broad range of biomarkers even when it needs information about only a few genomes or biomarkers. Accordingly, DeLePeDa-SAPs according to embodiments of the invention through the inclusion of ethics board approvals, etc. are designed from the viewpoint that requests for data sharing are justified by a research project's objectives and limited to specific biomarkers. Accordingly, DeLePeDa-SAPs according to embodiments of the invention may filter personal data authorised by a user so that it matches the data associated with the credential of the requestor. For example, a researcher's credential for a specific research project may define what data they are permitted to acquire and DeLePeDa-SAPs according to embodiments of the invention may employ this within the wallet.

Further, DeLePeDa-SAPs according to embodiments of the invention provides for one-to-one mapping of biomarkers to cryptographic health credentials, thereby ensuring that research projects specify the biomarkers needed for the study. This granular level of health data management and credentialing means that users only need to share health information for the specific biomarkers a particular study needs (see for example FIG. 8). This is fundamentally different to the prior art where access to a user's medical records is all encompassing. In contrast, DeLePeDa-SAPs according to embodiments of the invention allow for example a user to share a single item of biomarker data, health data or other personal data.

Within DeLePeDa-SAPs according to embodiments of the invention a user may be able to determine whether, for example, the range of their biomarkers matches the requirements of a study. Whilst the solution is designed so that users share only the specific biomarker information needed for a given study where monetization of the user's personal data when shared in a secure manner without releasing their identity is available then the user may be encouraged to increase the range of personal data they have available so that users are encouraged to expand and/or maintain the validity of their personal data. For example, a study may require personal data over a predetermined period of time with a last date within a given timeframe. In this manner, for example, an organization may increase the number of users providing a specific item or items of personal data as the incentive is provided to the users. For example, every male over 50 verifying a prostate test in the past 2 years receives a reward which may provide a government with increased statistics. Alternatively, during a pandemic all users validating that they have been tested receive a reward or a healthcare facility may restrict access to individuals who do not provide an item of personal data, e.g. a negative test result for a contagious infection.

Within the prior art records provide an audit trail offering evidence that a transaction has taken place, a requirement has been met, or an entitlement or claim exists. The availability of an audit trail increases overall trust in the operation of a system and provides assurance that all actors are compliant with operating rules and behaving honestly. At the same time, an audit trail may generate what the inventors refer to as “digital exhaust”, namely data that inadvertently reveals personally identifiable information about an individual. In the context of health research, an important audit trail surrounds users' consent to sharing of their health data. Keeping an audit trail of consents by recording them on the blockchain introduces a potential barrier to trust, however, since this makes it possible for third parties to observe the studies in which an individual is participating. Even though individuals' real world identities are masked by the use of DIDs, and the data may be encrypted, the design methodology of DeLePeDa-SAPs according to embodiments of the invention anticipates the risk of correlation, decryption (e.g., via quantum cryptoanalysis) or other techniques that may inadvertently reveal sensitive personal health information.

Accordingly, to avoid this risk and provide users with greater confidence that their interactions with researchers (and their health status) will not be revealed while still supporting the trust that an audit trail of consent delivers. Accordingly, DeLePeDa-SAPs according to embodiments of the invention can provide evidence of consent and the associated audit trail cryptographically and without the need to record information on the blockchain. DeLePeDa-SAPs according to embodiments of the invention may record and keep only Public DIDs, data schemas, credential definitions, and revocation registries on the blockchain. This design feature within DeLePeDa-SAPs according to embodiments of the invention is achieved by having individual data holders make a credential request to the other party, e.g. the researcher when considering Case 1 as an example, for a “consent enablement” credential. Thus, in considering Case 1 the researcher, not the individual, must send the consent enablement credential because, in the blockchain frameworks employed by DeLePeDa-SAPs according to embodiments of the invention, an individual cannot issue credentials. This is because issuers of credentials must store their public DIDs on ledger so that they may be used in cryptographic proofs of verifiable claims made on the basis of an issued credential. If individuals' DIDs were stored publicly on ledger for use in validation of claims relating to their consent, this would cause a potential risk of identification and could be out of compliance with privacy laws (e.g., GDPR). Accordingly, consider Case 1 the researcher then issues the consent enablement credential, which includes the terms and conditions of consent for the study as a cryptographic “consent enablement” credential with the SSI framework, which may be in human readable form. The cryptographic consent enablement credentials are stored in the individuals' wallets for later reference.

Individuals may within DeLePeDa-SAPs according to embodiments of the invention be given time to review the terms and conditions in human readable form either as provided or as rendered by a front end GUI, before the researcher (Case 1) issues a proof request as a means of indicating the individual's consent. If they consent to the terms and conditions, individual data owners will send back a proof of consent in response to the researcher's proof request, which includes a digitally signed hash of the terms and conditions. It is important to note that personal data within DeLePeDa-SAPs according to embodiments of the invention is not sent back with the proof of consent, as the proof of consent is kept in an audit data registry as evidence of the consent. If the data were bundled with the consent proof, this would result in accumulation of personal health information, potentially in clear text, thereby creating a security vulnerability and a potential privacy law violation.

Following proving of the consent, the researcher then verifies the information sent back from the individual data owner, validating the proof. The inventors anticipate that the consent proof will be stored by the researcher in an “audit data registry.”. If subsequently, a third party requires proof of consent to validate that the researcher obtained the proper consent from the user, the researcher is able to present the consent proof, which can always be cryptographically (re)validated to prove its authenticity. Once the researcher has received the consent proof, a “data sharing” credential offer is made to the individual data owner. The individual accepts the offer and is sent the credential, which also contains a digitally signed copy of the consent proof. An individual thus has their own copy of the validated proof. At this point the researcher requests a proof from the individual to trigger the data sharing. The individual then sends the agreed biomarker data as a revealed attribute in the proof. The researcher validates the proof and accepts the data. This triggers the final credential offer from the researcher for a “reward” credential, which the individual requests and is sent as their reward for sharing their data for research purposes. In this way a cryptographically provable audit trail is created without resorting to recording individuals' interactions with researchers on ledger. It would be evident that whilst the above description is provided with respect to Case 1 such a process flow may be employed with Case 2 and other business cases.

Audit Data Registry

As noted above in order to support within DeLePeDa-SAPs according to embodiments of the invention and retain the advantageous privacy-preserving and self-sovereign capabilities of credential-based tokenized systems while supporting the need to keep evidence for accountability, audit, legal and compliance purposes, it is necessary to add a new capability to SSI credential-based tokenized systems. This new capability being referred to by the inventors as a “audit data registry”.

An audit data registry according to embodiments of the invention comprises technical components, data structures, and process flows. In order to ease understanding the following description the terminology employed by the inventors is based upon the definitions of the World Wide Web Consortium (W3C) Verifiable Credentials Working Group. Accordingly:
Credential: A credential is a set of claims made by an issuer. A verifiable credential is a tamper-evident credential that has authorship that can be cryptographically verified.
Issuer: A role an entity can perform by asserting claims about one or more subjects, creating a verifiable credential from these claims, and transmitting the verifiable credential to a holder.
Holder: A role an entity might perform by possessing one or more verifiable credentials and generating presentations from them.
Verifier: The entity verifying a claim about a given subject.
Proof Request: The request for one or more verifiable credentials, issued by one or more issuers, held by one holder.
Proof Presentation: Data derived from one or more verifiable credentials, issued by one or more issuers, that is shared with a specific verifier.
Proof Verifications: The evaluation of whether a verifiable credential or verifiable presentation is an authentic and timely statement of the issuer or presenter, respectively.

Audit data registry Technical Components: One of the technical components of an audit data registry is a tamper-resistant and immutable store of proof data. Whenever credentials are exchanged, the corresponding proof requests, proof presentations, and proof verifications are sent to the audit data registry store.

Audit data registry Data Structures: Each sequence of proof requests, proof presentations, and proof verification based on a specific verifiable credential should be captured as a single record in the store of proof data and the data structure of each such sequence of proofs should contain specific attributes (e.g., credential identifiers and proof identifiers) to link the proofs back to their underlying credential and to associate the proofs with one another. This establishes what is referred to as an “archival bond” amongst these data structures, which would otherwise be passed as unconnected messages between communicating peers, and captures them into and preserves them in the data store for a required period of time (e.g., the period of time for which the evidence may be required by law, regulation, or audit purposes) in a manner that allows for the preservation of their authenticity and integrity and enables them to serve as trustworthy evidence of the facts to which they attest.

Audit data registry Process Flows: With regard to the protocol, according to embodiments of the invention described and presented here, only the verifier communicates with the audit data registry directly. The credential holder establishes a connection with the verifier and completes a handshake, after which the verifier transmits all corresponding proof data to the audit data registry. The verifier ensures that proof data is complete and consistent before it is stored persistently, and is, moreover, incentivized to do so in order to be in a position to demonstrate compliance, respond to audits, etc.

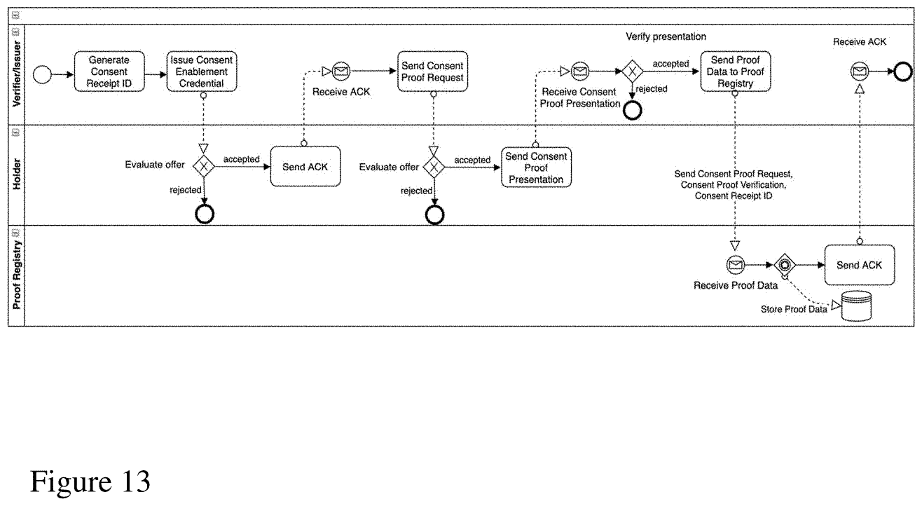

Prototype Implementation of an audit data registry: The inventors have implemented a prototype of the audit data registry in the context of verifiable health credentials and present the process flow in the following Business Process Method Notation (BPMN) diagram according to an embodiment of the invention in FIG. 13.

Consider, the scenario with two entities, Alice (Issuer/Verifier) and Bob (Holder). For example, Alice could be a corporation running medical trials, whereas Bob could be an individual who is interested in participating in the said trial by sharing his verifiable health data. Prior to verifiable health data being shared, however, consent to participate in the medical trial has to be established. To do that, Alice generates a consent enablement credential, which has a specific consent receipt ID as one of its attributes, in order to be able to record that consent for sharing particular health data was given. The hashlink to the terms and conditions of consent is also included in order to ensure that consent only applies to that particular case. Once this credential is generated, Alice sends the consent enablement credential to Bob. Bob can then either accept or reject the credential. If accepted by Bob, Alice can then send a consent proof request to Bob, which, upon acceptance of the request and presentation of the proof, signals Bob's consent to participate in the medical trial.

To capture a record of the proof request, Alice's client keeps a copy of the request and will send it to the audit data registry's proof data store later on with other proof data.

Subsequently, Alice sends Bob a proof request asking him to share his consented personal health information. Bob sends this to Alice in a proof. The proof contains ⅓ of a secret key generated using cryptographic techniques at time of issuance of a health data credential by the issuer of said credential. ⅓ of the key is retained by the issuer and ⅓ of the key is retained by the owner of the credential in their wallet. At the time that Alice receives Bob's health data, Alice's client receives the data and also sends to the audit data registry a copy of the proof from Bob (without the PII) and along with the ⅓ secret key in order to be able to unlock the identity of Bob, should this be necessary in case of compliance requirement or emergency, by matching ⅔ secret keys using cryptographic techniques. In this way audit, accountability and compliance requirements can be met without comprising the privacy of data owners.

All such data is stored in a record discoverable by the consent receipt ID. The following steps in the process flow include the proof presentation, and the proof verification, each of which will also contain the consent receipt ID as one of their attributes and be stored in the audit data registry data store.

In this manner, a history of the issued proof is stored reliably, persistently, and for as long as required (e.g., by law or regulation) and in a tamper-resistant manner. Meeting these criteria can be of importance, particularly in light of regulatory compliance, such as the US regulations on electronic documents and signatures (see for example 21 CFR Part 11) which specify the requirements that need to be met in order to assure that electronic records are trustworthy and admissible as legal evidence.

In the exemplary embodiment depicted in FIG. 13, the captured and stored sequence of proofs provide reliable and authentic evidence that consent was given to participate in the medical trial and exchange health information, regardless of whether the holder keeps the original peer-to-peer connection alive. This permits third parties who may have legal, audit or accountability functions to retrieve the proof data from the audit data registry's data store (e.g., by the consent receipt ID) in order to:
Establish for each instance for which verifiable data sharing is requested that data sharing is consented.
Establish for specific data shares that usage is consented.
Verify the terms and conditions of consent agreed to.
Establish that the terms of consent at an initial time t₁ have not been altered at a subsequent time t₂.
Unlock the real identity of a data owner (sharer of data) should this be necessary for legal or regulatory purposes, or in case of emergency without comprising the privacy of the data owner (sharer)

Having proof data stored in the registry allows auditors to verify its validity, even in the case of incomplete data, e.g., in the case of deleted credentials by the holder. For an exemplary hyperledger implementation a proof presentation could be validated by an auditor (verifier) by simply retrieving and revalidating the proof request and proof, which, without an audit data registry, might not be accessible.

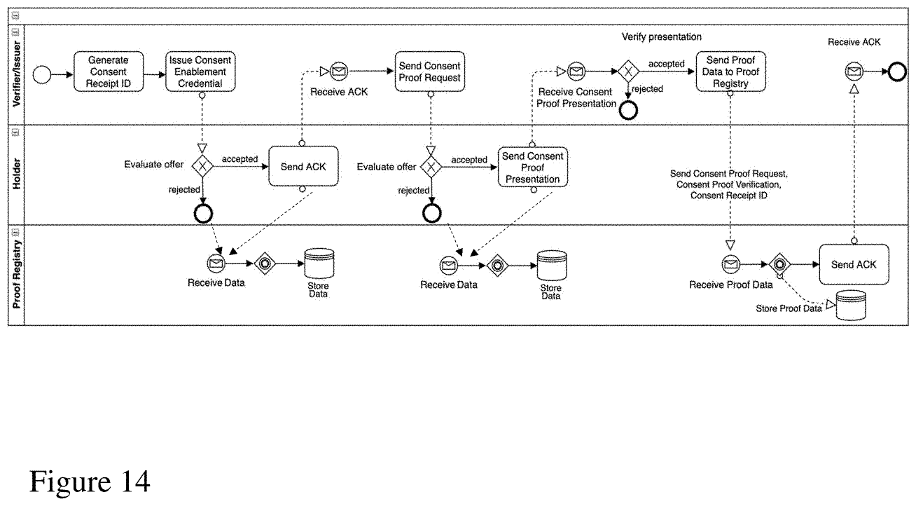

Within FIG. 13 the audit data registry only stores the final approval from the verifier/issuer. However, referring to FIG. 14 there is depicted a BPMN diagram for an audit data registry according to an embodiment of the invention where the intervening decisions are also stored within the audit data registry. Accordingly, the acceptance/rejection of the evaluated offer with respect to the issued consent enablement credential by the holder and the subsequent acceptance/rejection of the evaluated offer by the holder of the sent consent proof request are also stored within the audit data registry. It would be evident that other steps of the exemplary process depicted in FIGS. 13 and 14 respectively may also be stored within the audit data registry such as the acceptance/rejection of the verified presentation.

In order to assess the efficacy of the exemplary implementation of the audit data registry the inventors performed experiments conducted through a simulated process of health data sharing with consent. Specifically, during this process, an individual who has eligibility and agrees to participate in a medical trial receives the consent enablement credential from the researcher who is requesting data for a medical trial.

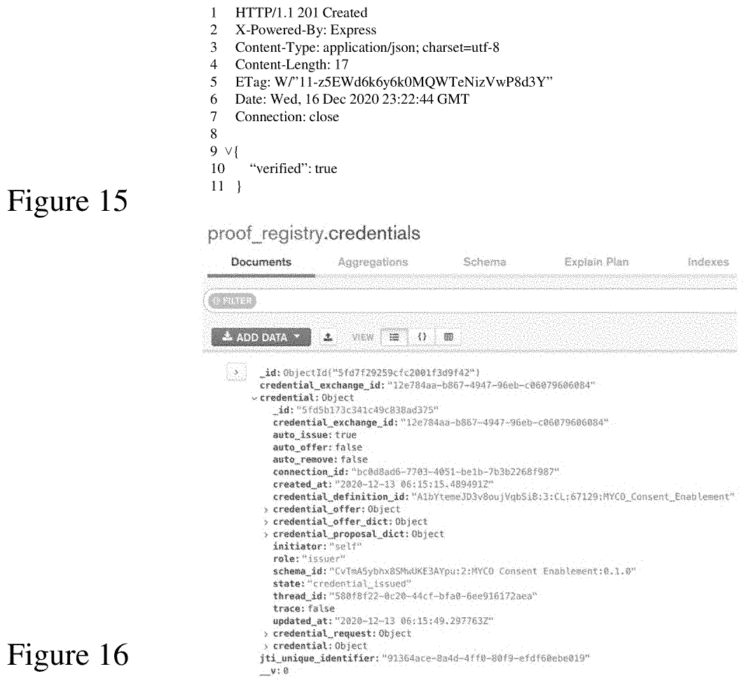

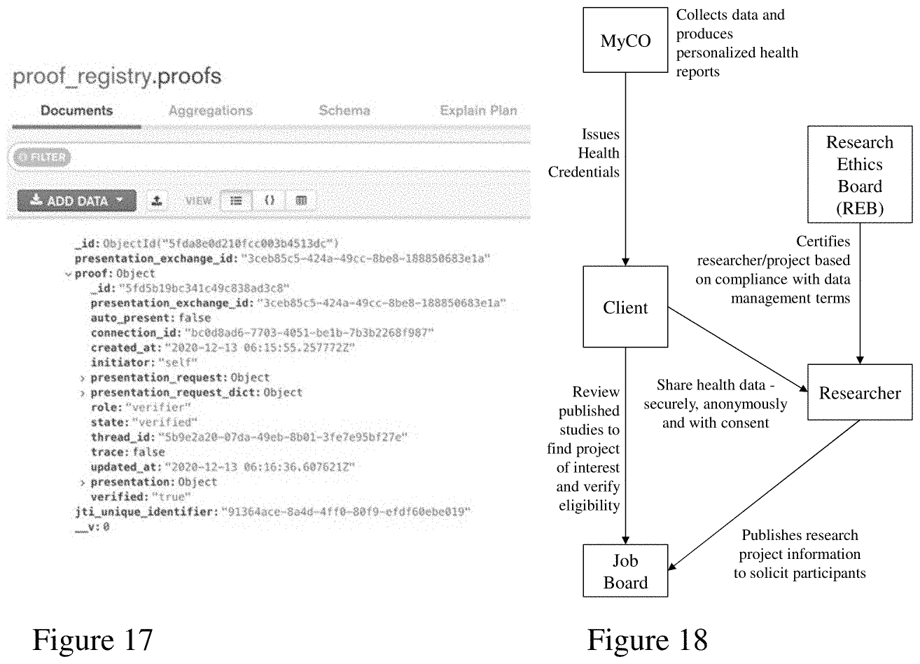

The individual provides consent by presenting a consent proof based on the consent enablement credential. An example of a stored credential according to an embodiment of the invention is depicted in FIG. 16. It is worth noting that the ‘jti unique identifier’ represents the specific consent receipt ID. When the auditor needs to audit the consent proof, they can find the proof from the audit data registry database. An example of such a proof as stored within an audit data registry according to an embodiment of the invention, this being depicted within FIG. 17, through the identity and use its presentation and presentation request attributes to re-verify the proof. A re-verification would only be successful with the outline original payloads, as otherwise, the signatures would differ, and the validation would fail. FIG. 15 depicts a response when this proof has been re-verified.

Exemplary proof registries according to embodiments of the invention as implemented by the inventors also provide the evidence needed to establish for specific data shares that a specific owner of the data (i.e., legally identifiable credential holder) consented to its usage without violating the privacy preserving principles of operation of SSI protocols through the application of cryptographic techniques for the splitting and sharing of secret keys distributed between the an issuer, a data owner and a recipient of shared data. Such linkages are necessary to establish accountability and prevent non-repudiation for legal reasons and accordingly may be implemented within other exemplary embodiments of the invention.

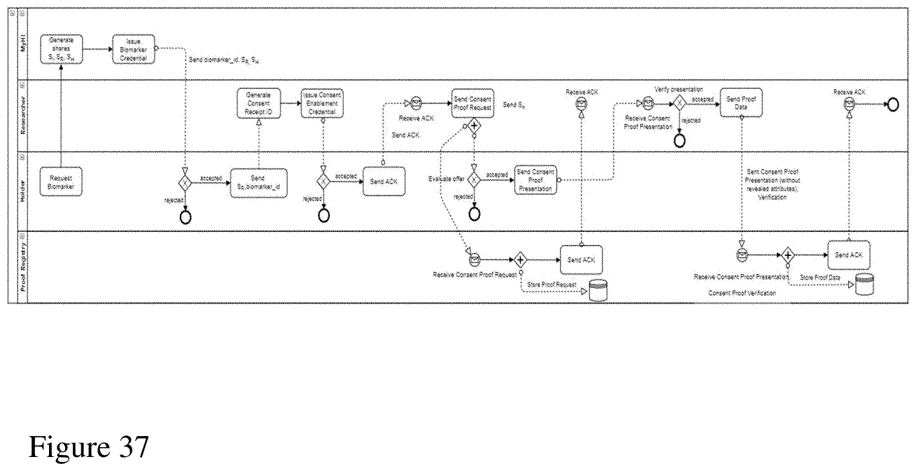

In some instances, the explicit authorisation of an individual to have data relating to their specific identity released may be required. Such an instance being depicted within FIG. 37 which depicts an exemplary BPMN diagram for a handshake with respect to a holder and a researcher (requester) relating to providing data relating to the holder to the researcher according to an embodiment of the invention. Accordingly, the exemplary process flow depicted in FIG. 37 comprises a flow incorporating an initial request by the holder to and response from MY Hyperledger Indy (MYHI) followed by exchanges between the holder with both the researcher (seeking access to the holder's data e.g. biomarker) and the audit data registry (proof registry).

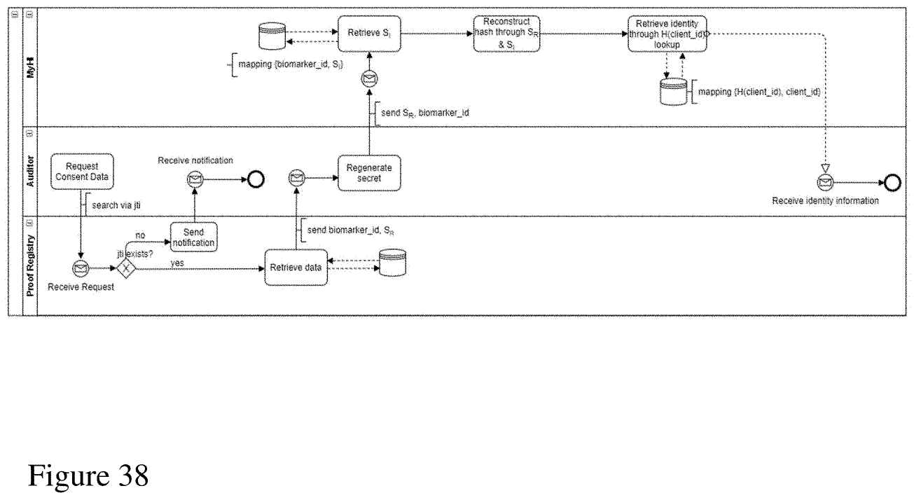

Further, referring to FIG. 38 there is depicted an exemplary BPMN diagram for retrieval of identity information by an auditor relating to a consent according to an embodiment of the invention. Accordingly, the exemplary process flow depicted in FIG. 38 comprises a flow incorporating an initial request by the auditor to and response from the audit data registry (proof registry) followed by communications by the auditor to and from the MY Hyperledger Indy (MYHI) resulting in provisioning of the identity information.

Exemplary Decentralized Ledger Personal Data Process flows

Within the preceding sections the methodologies, concepts, and associated infrastructure, registries for Decentralized Ledger Personal Data (DeLePeDa) Systems, Applications and Platforms (DeLePeDa-SAPs) have been described and presented. Within this section exemplary process flows associated with these DeLePeDa-SAPs according to embodiments of the invention are described and presented. Within the following description an enterprise referred to as “MyCO’, for example, Molecular You, acts as a “source” of data by issuing health data as Verifiable Credentials. My CO clients MyCO Clients store their Health Credentials in a mobile wallet. Within one use scenario researchers publish information about studies for which they wish to solicit participants for through a “Job Board.” Studies are “certified” by a Research Ethics Board (REB). If a Client is interested and can demonstrate eligibility, they are connected with the Researcher for secure exchange of Consent and Health Data. Researchers can demonstrate proof of Consent for all collected data. Within embodiments of the invention MyCO, the REB, Researchers and the Job Board run instances of a hyperledger cloud agent.

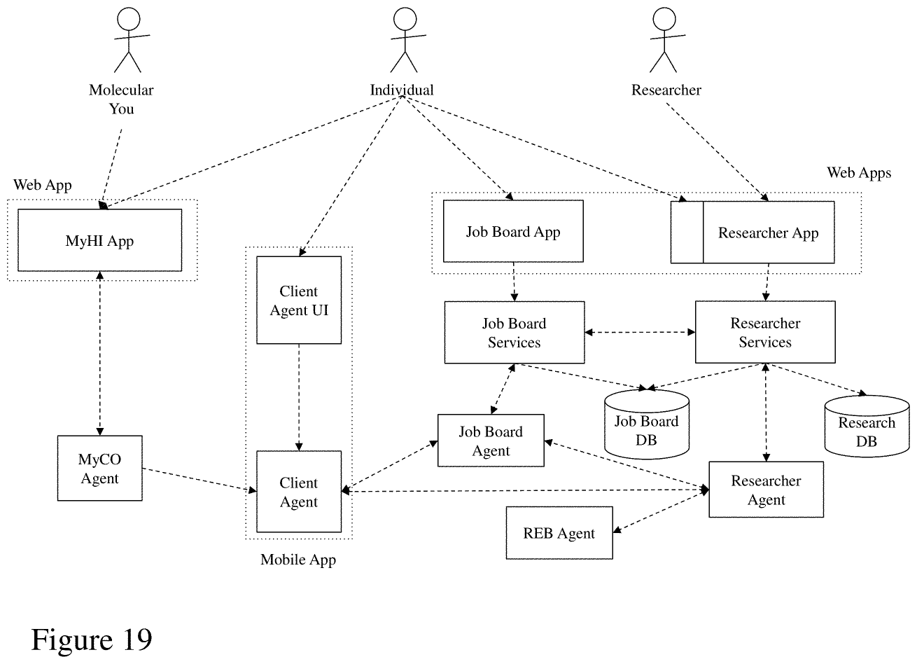

FIG. 18 depicts a logical process flow and architecture for a DeLePeDa -SAP according to embodiments of the invention whilst FIG. 19 depicts an exemplary architecture for a for a DeLePeDa -SAP according to embodiments of the invention.

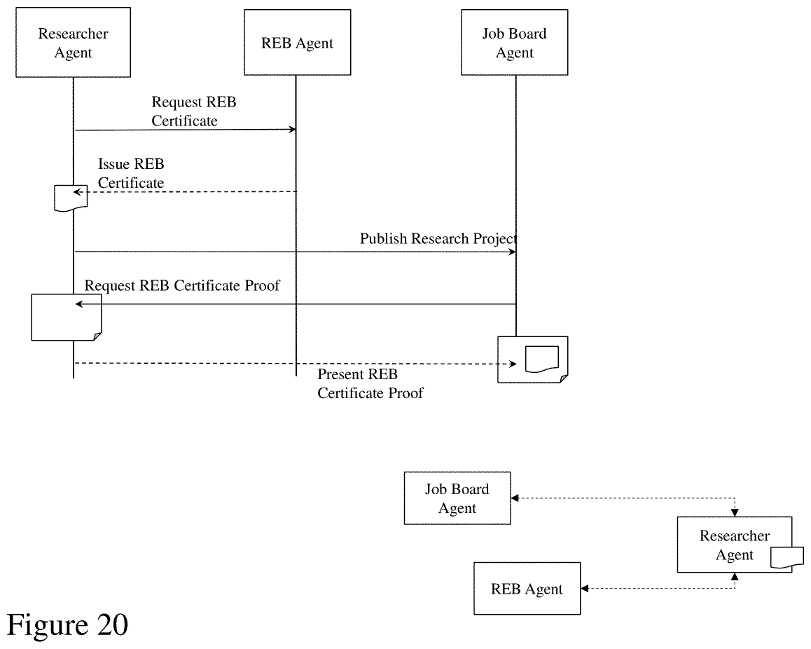

Now referring to FIG. 20 there is depicted an exemplary process flow for a researcher project set up and research ethics board certification exploiting DeLePeDa -SAPs according to embodiments of the invention. Accordingly:
Researchers post project information, including:
   Data requirements (biomarkers); and
   Data use, location, etc.
Certification request is sent to REB:
   Credential Proposal, including a hashlink to the project information;
   REB issues a Credential; and
Job Board requests “Proof of Compliance” prior to posting the Project.

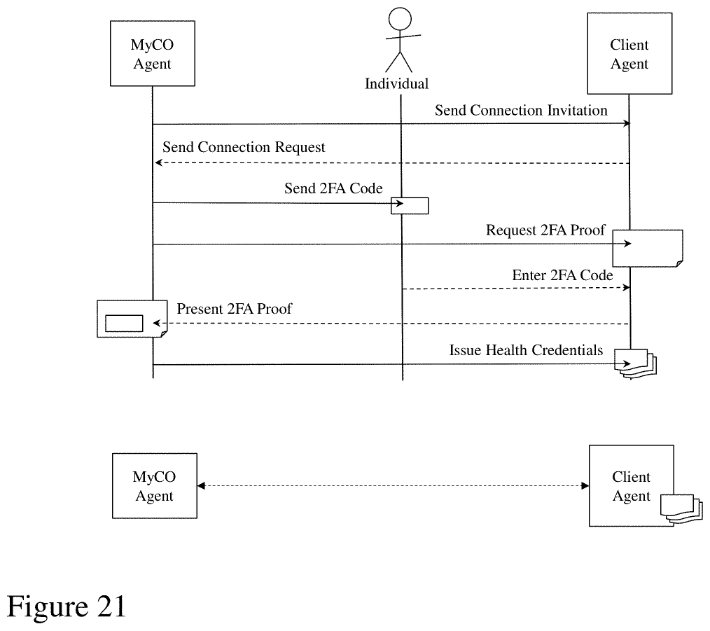

Referring to FIG. 21 there is depicted an exemplary process flow for a user connection and credential receipt within DeLePeDa -SAPs according to embodiments of the invention. Accordingly:
Clients connect through an existing web portal—MyHI:
   Can choose to “opt-in” to MyPDx;
MyCO issues a connection to the Client's mobile wallet:
   MyHI sends a 2-factor code to the Client to ensure the connection does not get hijacked; and
Once the connection is established MyHI issues Health Credentials to the Client's wallet:
   Optionally issues a MyCO “Identity” credential as well.

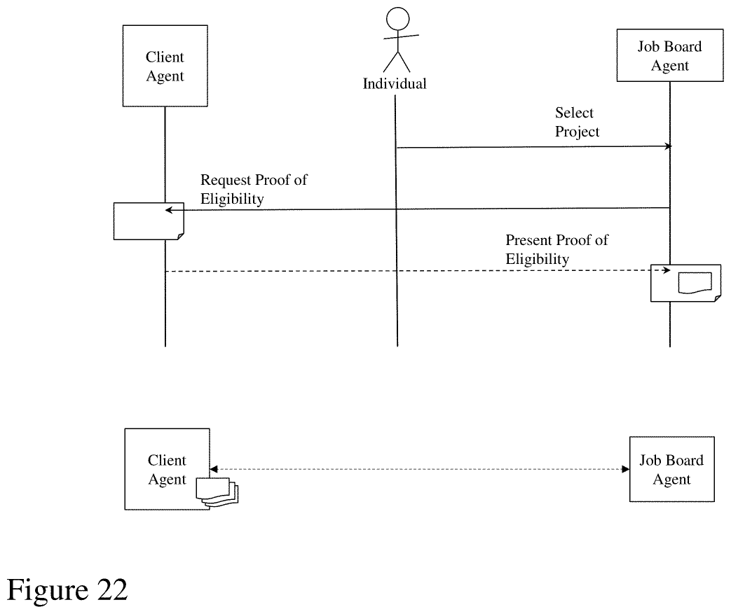

Referring to FIG. 22 there is depicted an exemplary process flow for project eligibility proof within DeLePeDa -SAPs according to embodiments of the invention. Accordingly:
Project Eligibility is based on the data requirements of the Research Project:
   Biomarker type and level;
   Client responds with a Zero Knowledge Proof (ZKP):
   This may depend on hyperledger software development kit (SDK) functionality;
Job Board does not track any user information/activity
   Potential to “login” to the job board using a MyCO Identity Credential.

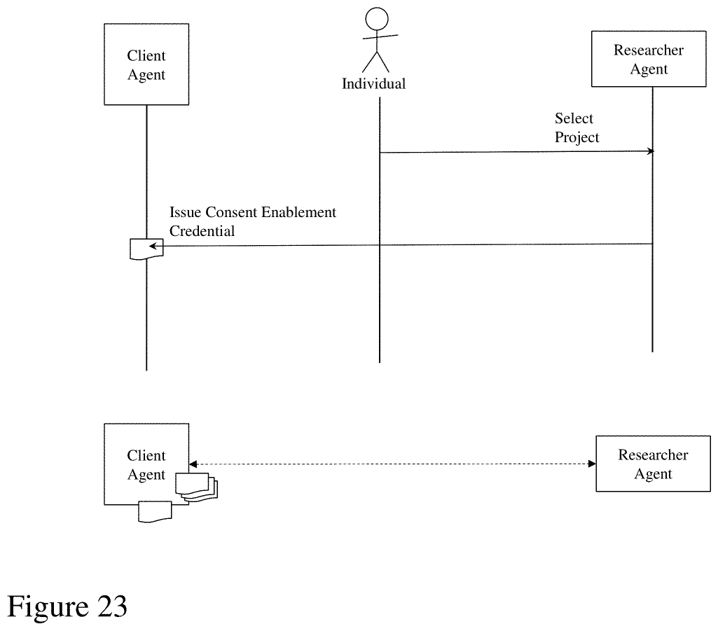

Referring to FIG. 23 there is depicted an exemplary process flow for a client receiving consent credentials from a research within DeLePeDa -SAPs according to embodiments of the Invention. Accordingly:
Secure connection is established between Client and Researcher agents;
Consent information is issued as a Verifiable Credential:
   We call it a “Consent Enablement Credential”;
   Follows Kantara Consent Receipt standards;
   Contains a hashlink to research project information and consent terms;
Client acceptance of the Consent Credential does not (yet) indicate Consent to Share Data; and
Credential contains a unique identifier.

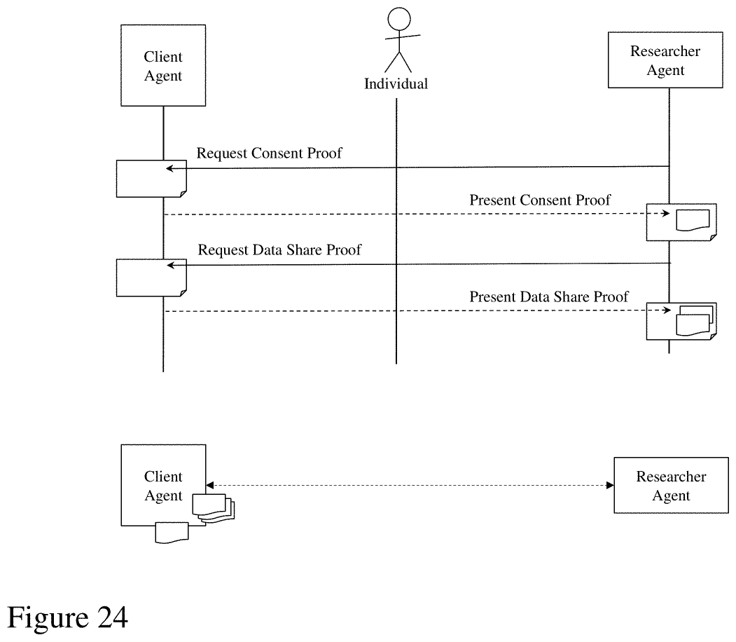

Referring to FIG. 24 there is depicted an exemplary process flow for a client consenting with proof and sharing health data with proof within DeLePeDa -SAPs according to embodiments of the Invention. Accordingly:
Researcher asks the Client to provide two proofs:
   Proof of Consent:
      Includes attributes from the Consent Enablement Credential
   Proof of Health Data:
      Includes the “id” from the Consent Credential and the requested health data;
The two proofs are linked by the Consent id;
The consent terms are contained in a hashlinked document;
   This same hashlink is in the REB credential issued to the researcher for this project.

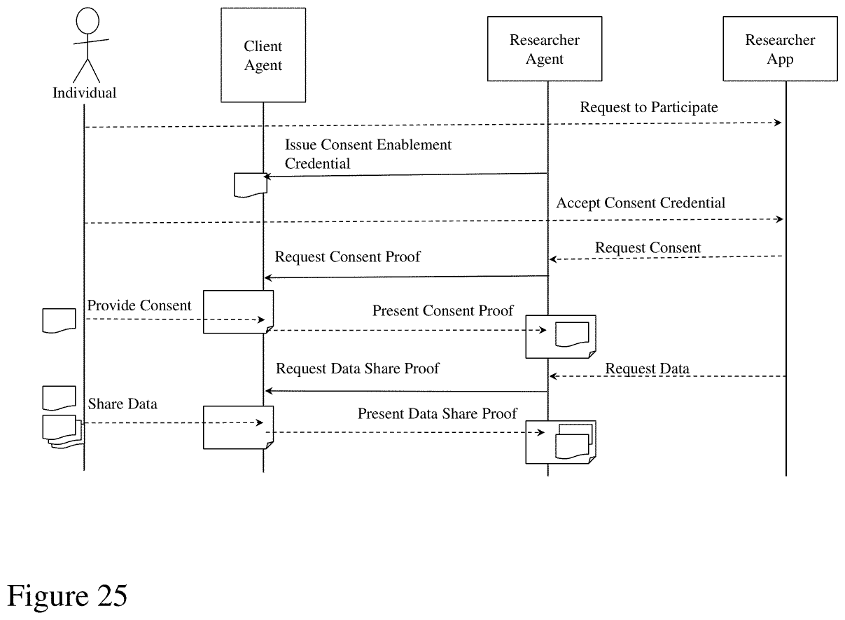

Referring to FIG. 25 there is depicted an exemplary process flow for consent receipt within DeLePeDa-SAPs according to embodiments of the Invention. Accordingly:
Researcher will maintain a library of received proofs in a “Audit data registry”;
Use to demonstrate compliance to an Auditor;
Two proofs are received (linked by the Consent Credential ID):
   Proof of Consent;
   Proof of Shared Data;
Auditor can verify consent without access to health data;
“Chain of proof” can verify:
   Health data was issued by a known issuer;
   Consent and data were provided by the same “holder”;
   All data is shared with Consent;
   Consent terms have not changed (hashlink); and
   Proofs are linked by a unique consent id.

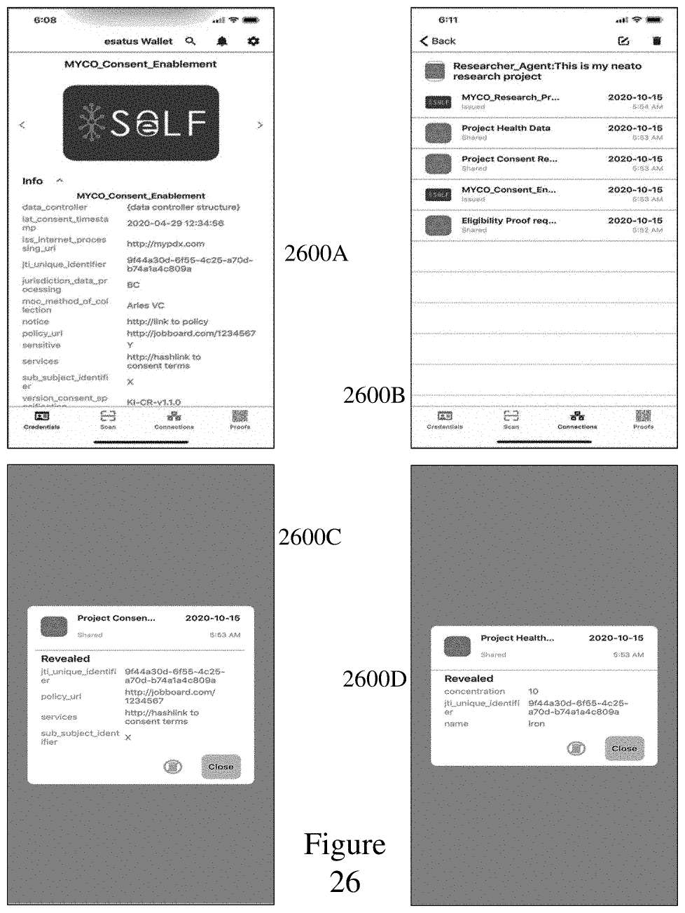

Referring to FIG. 26 there are depicted first to fourth Screenshots 2600A to 2600D of a software application depicting the consent receipt and proofs as described with respect to embodiments of the invention.

Within embodiments of the invention the information describing the consent is issued as a Verifiable Credential (VC) from the Researcher to the Client (Research Subject). This may contain, for example, a unique identifier (e.g., a “jti_unique_identifier”) and a hashlink to project/consent details (currently a pdf) and may follow the Kantara specification. A Consent is provided as a “proof”. Consent and shared Health Data are provided in two separate proofs which are linked by the consent identifier, the unique identifier (e.g., “jti_unique_identifier”). Importantly, embodiments of the invention provide for Consent to be audited without revealing personal data. Further, revocation of consent may be established by a Client.

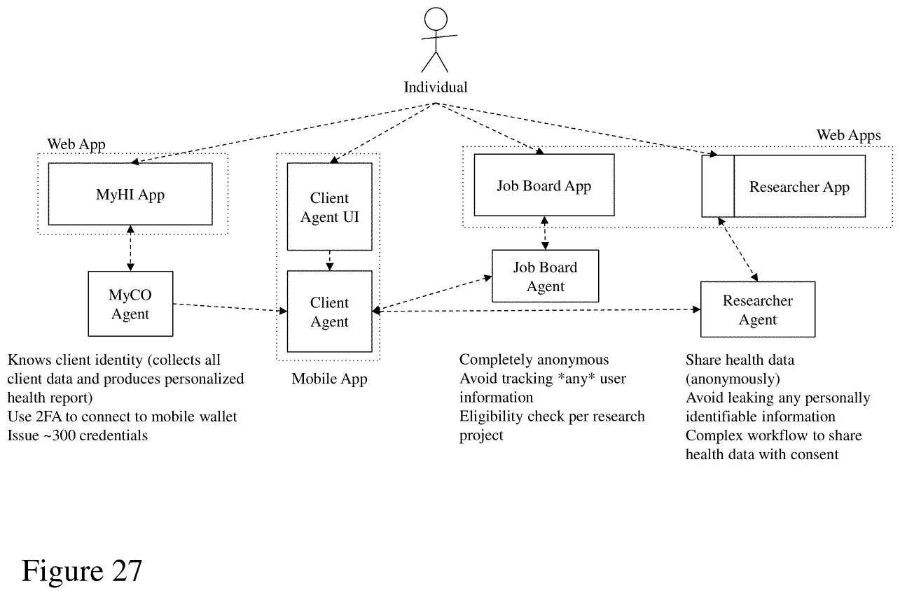

Referring to FIG. 27 there is depicted exemplary interactions within DeLePeDa -SAPs according to embodiments of the invention. Within embodiments of the invention described herein SSI applications are employed. Accordingly, the client interacts with:
MyCO—existing relationship and identity, existing application (e.g., MyHI), to issue health credentials;
Job Board—“anonymous” relationship, client can peruse studies and check eligibility without revealing any identity information; and
Researchers—share health data with consent; anonymous de-identified data

Amongst the challenges addressed by embodiments of the invention are:
Usability to provide a solution exploiting mobile and web applications;
Security and privacy in order to avoid “leaking” information; and
Working with generally available technology/functionality.

Other considerations for embodiments of the invention include, but are not limited to:
Governance frameworks;
VC-based workflows; and
A “tri-brid” architecture as the inventors refer to it with a hybrid mobile/cloud wallet for individuals combined with a web application.

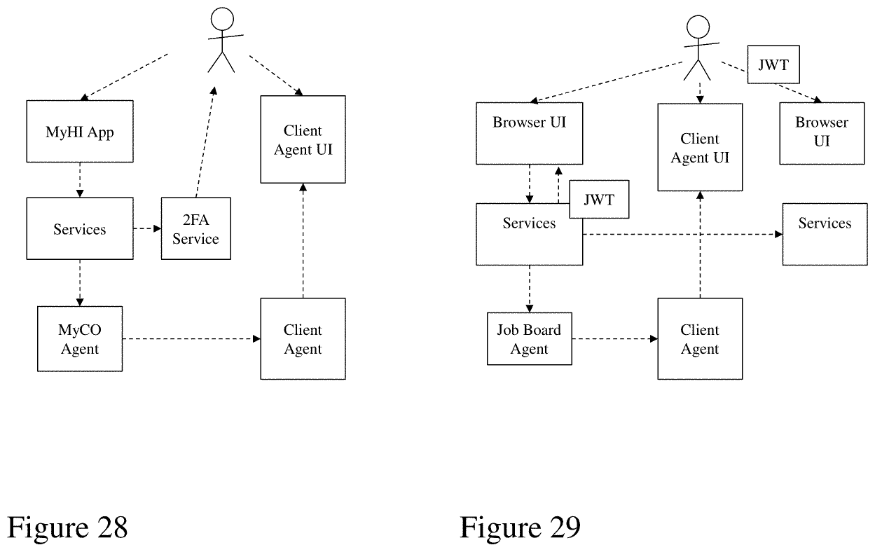

Referring to FIG. 28 there is depicted an exemplary process flow for issuing health credentials within DeLePeDa-SAPs according to embodiments of the Invention. Accordingly, the Client—MyCO interaction to issue health credentials utilizes:
Participants who are existing MyCO clients:
   Clients provide laboratory samples;
   MyCO produces a personalized health report;
   MyHI provides the clients with a web based portal to interact with MyCO;
MyCO client can enroll in the MyPDx service by:
   Establishing a connection between MyCO and client agents
   Using a two-factor authentication (2FA) code to ensure connection URL is not “hijacked”; and
   Client providing 2FA code using a “Self-Attested Proof.”
MyCO issues for example approximately 300 health credentials for the client based upon the processing of the laboratory samples; which includes:
   Biometrics; and
   Genetic information.

Referring to FIG. 29 there is depicted an exemplary process flow for a client browsing projects and choosing to enroll within DeLePeDa -SAPs according to embodiments of the Invention. Accordingly:
Client can browse projects and check eligibility:
   Eligibility check performed via Zero Knowledge Proof (ZKP);
   Job Board does not track/record eligibility checks;
   State is recorded using local browser storage;
If user decides to “enrol” and share data with a researcher:
   Job Board produces a token which is sent to the Researcher application (via web service call) and also sent to the client;
   Re-directs to an enrolment page in the Researcher web UI; and
   Client provides the token to access the enrolment page; and
Connections and tokens are per project.

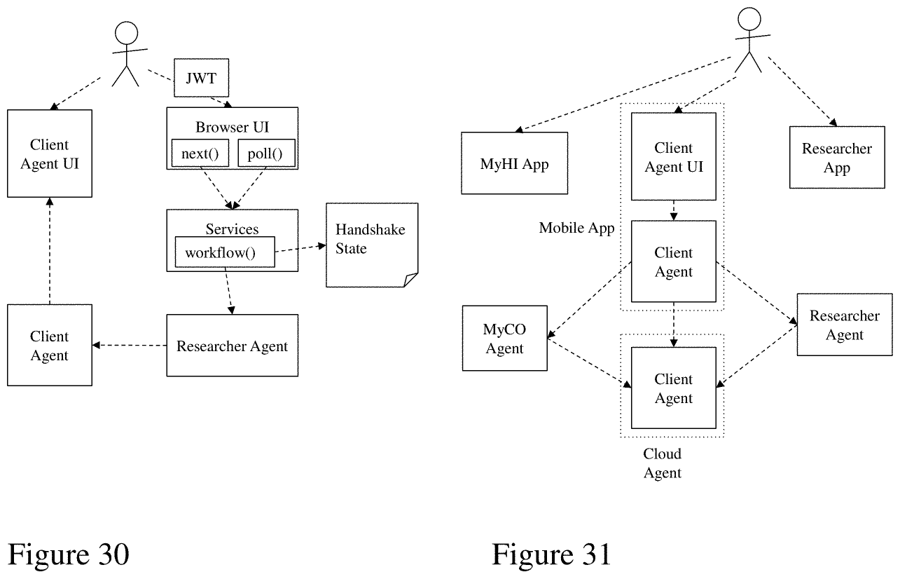

Referring to FIG. 30 there is depicted an exemplary process flow for client to researcher interactions for sharing data with consent within DeLePeDa -SAPs according to embodiments of the Invention. Accordingly:
A client interacts with a workflow in several steps:
   Establish agent connection;
   Demonstrate eligibility;
   Provide consent (2 steps within embodiments of the invention described but could be a single step or N>2 steps where N is a positive integer);
   Share health data
A browser UI provides:
   Display workflow state and allow user to “next( )” step;
   Uses a “per project” token to identify the workflow instance;
A Client Agent UI provides:
   Receipt of credentials;
   Provides proof(s); and
   Provides usable experience while protecting the security of the user's information.

As noted above initial prototype embodiments of the invention provide a user with a solution requiring the user to switch between mobile and web applications with a consent/data sharing workflow comprising of five steps. Accordingly, it is important that the solution provides security and privacy in order to how to avoid leaking information. In order to minimize this an agent sends status to the web UI to display status to the user and allows the user to interact with it.

It is also important for embodiments of the invention to provide the appropriate governance frameworks for the network of data issuers (MyCOs), consumers (e.g., Researchers) and compliance verifiers (e.g., REBs) within the different use cases. Accordingly, it is important that the governance framework defines who can participate in the network and with what role as well as determining what defines the Trust in the network. The use of VC-based workflows allows for configuration of workflows exploiting web and/or mobile applications whilst the tri-brid architecture exploits a hybrid mobile/cloud wallet for individuals combined with a web application.

Referring to FIG. 31 there is depicted an exemplary SSI architecture within DeLePeDa-SAPs according to embodiments of the Invention. Accordingly, the user interacts directly with a mobile agent which authenticates through the use of cloud agent allowing bulk data to be issued to the cloud agent, e.g., Molecular You releases approximately 300 credentials (each credential being an item of health data). It would be evident that by issuing a large number of credentials the embodiments of the invention prevent hacking a single database to access all health information for an individual.

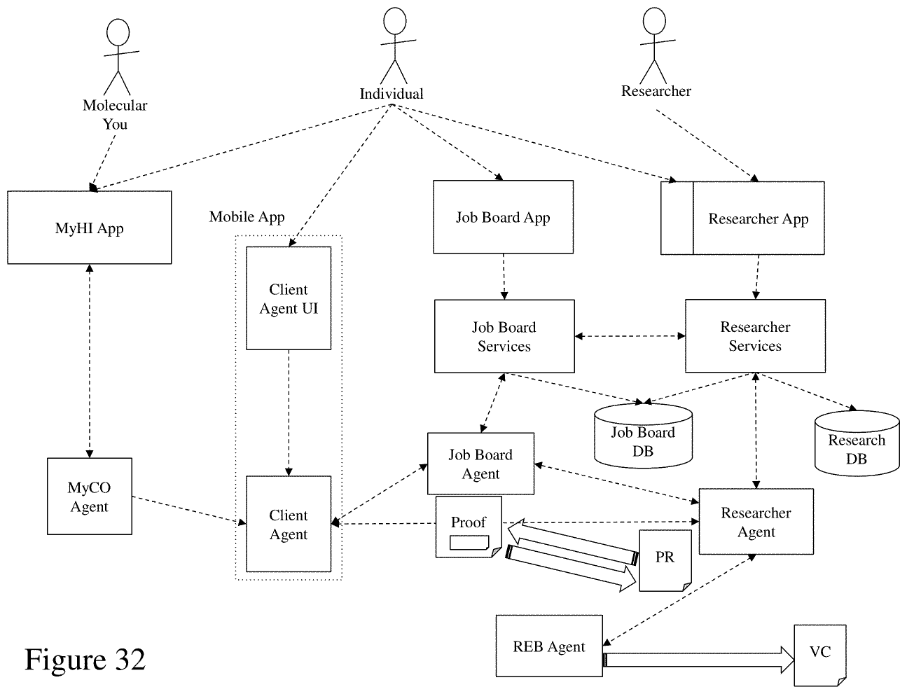

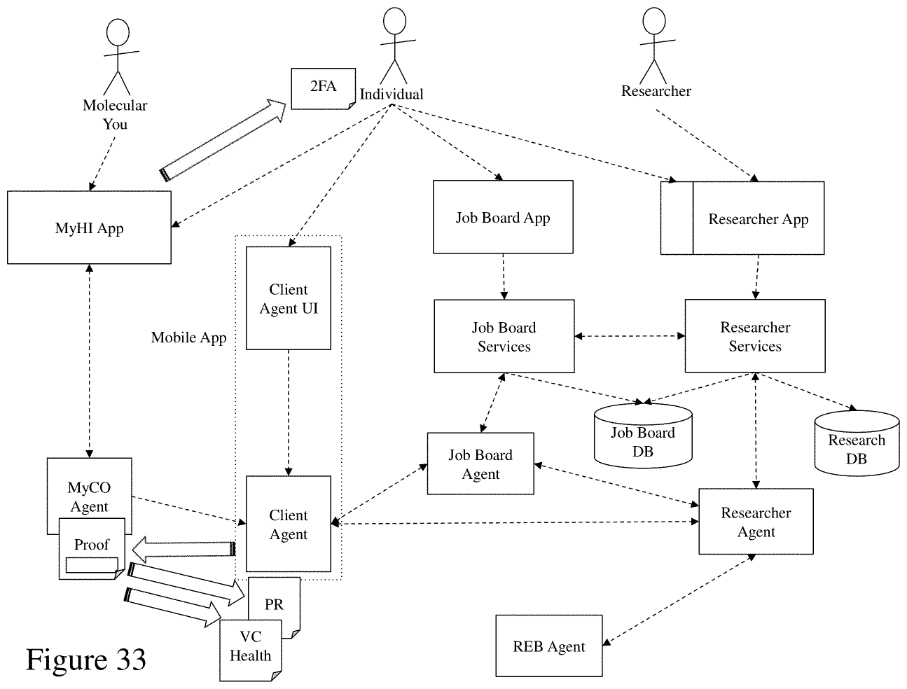

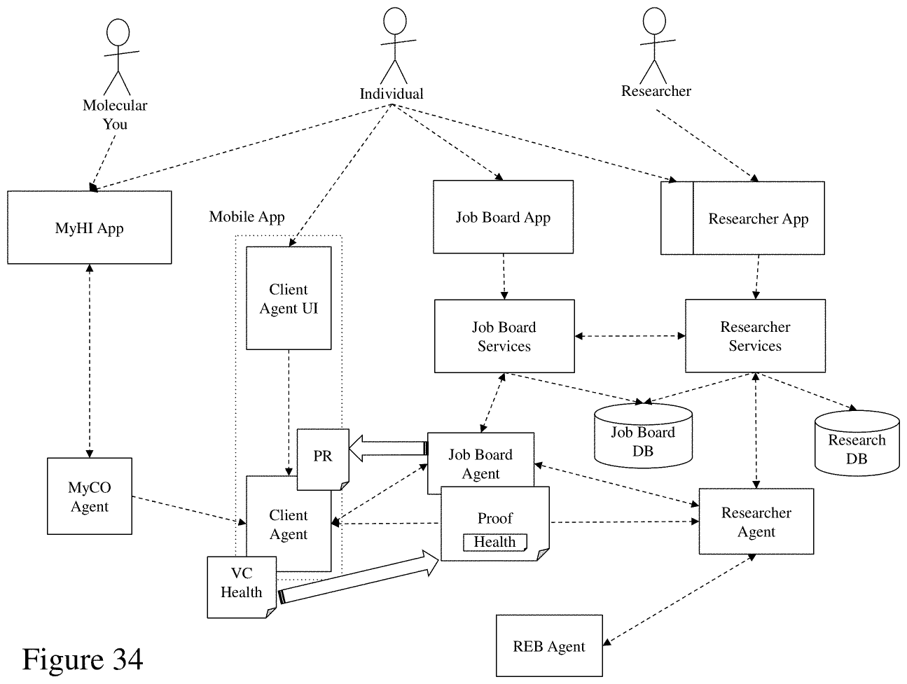

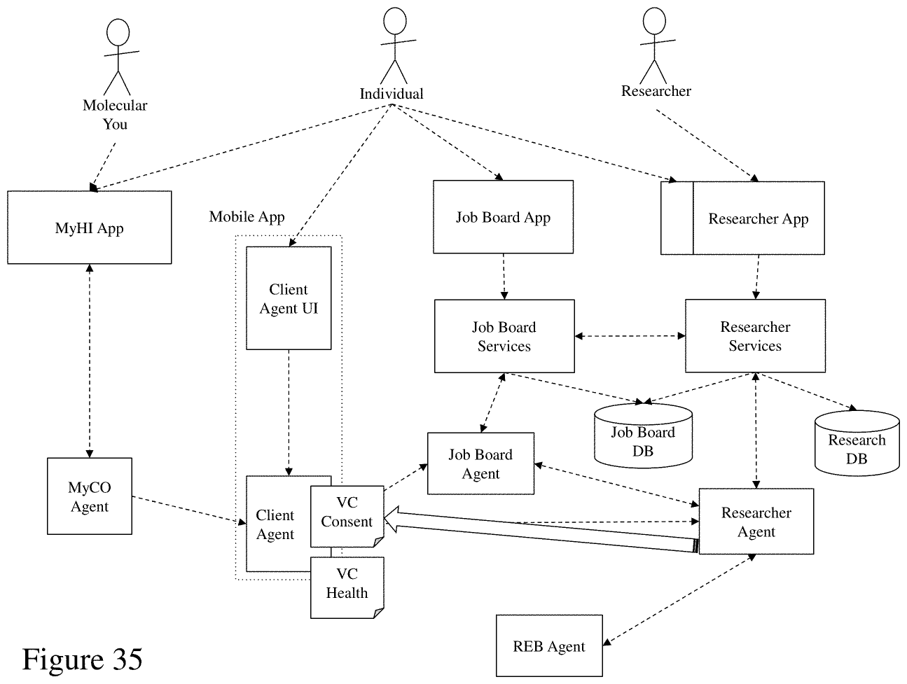

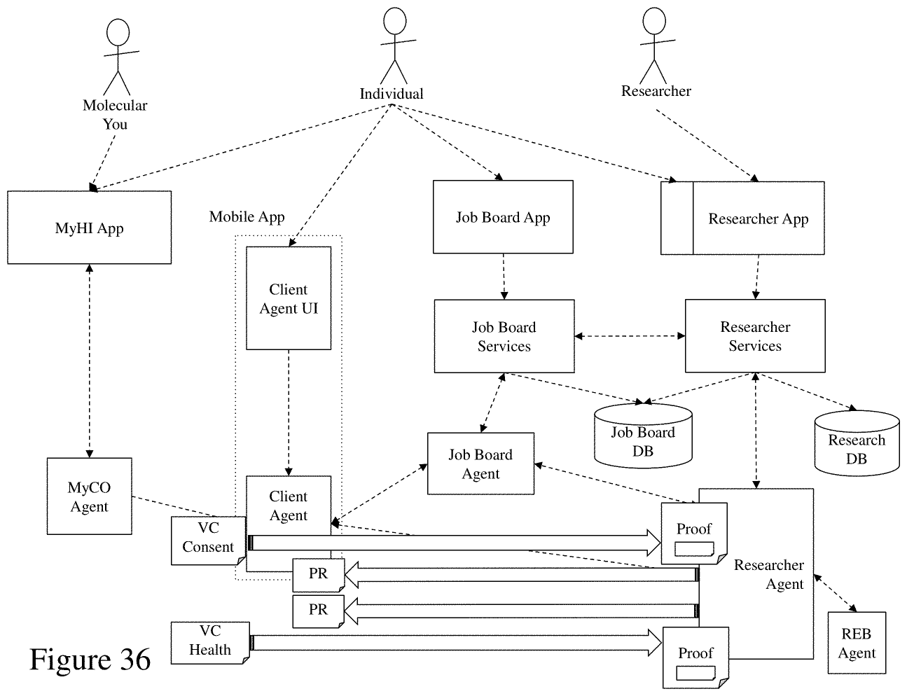

Referring to FIGS. 32 to 36 there are depicted exemplary architectures with messaging flow(s) within DeLePeDa-SAPs according to embodiments of the invention for the following:
project setup and research ethics board certification;
client connection and credential receipt;
project eligibility proof;
consent credential receipt; and
consent and sharing health data (both with proof).

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps, and means described above may be done in various ways. For example, these techniques, blocks, steps, and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages and/or any combination thereof. When implemented in software, firmware, middleware, scripting language and/or microcode, the program code or code segments to perform the necessary tasks may be stored in a machine readable medium, such as a storage medium. A code segment or machine-executable instruction may represent a procedure, a function, a subprogram, a program, a routine, a subroutine, a module, a software package, a script, a class, or any combination of instructions, data structures and/or program statements. A code segment may be coupled to another code segment or a hardware circuit by passing and/or receiving information, data, arguments, parameters and/or memory content. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor and may vary in implementation where the memory is employed in storing software codes for subsequent execution to that when the memory is employed in executing the software codes. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to, portable or fixed storage devices, optical storage devices, wireless channels, and/or various other mediums capable of storing, containing, or carrying instruction(s) and/or data.

The methodologies described herein are, in one or more embodiments, performable by a machine which includes one or more processors that accept code segments containing instructions. For any of the methods described herein, when the instructions are executed by the machine, the machine performs the method. Any machine capable of executing a set of instructions (sequential or otherwise) that specify actions to be taken by that machine are included. Thus, a typical machine may be exemplified by a typical processing system that includes one or more processors. Each processor may include one or more of a CPU, a graphics-processing unit, and a programmable DSP unit. The processing system further may include a memory subsystem including main RAM and/or a static RAM, and/or ROM. A bus subsystem may be included for communicating between the components. If the processing system requires a display, such a display may be included, e.g., a liquid crystal display (LCD). If manual data entry is required, the processing system also includes an input device such as one or more of an alphanumeric input unit such as a keyboard, a pointing control device such as a mouse, and so forth.

The memory includes machine-readable code segments (e.g. software or software code) including instructions for performing, when executed by the processing system, one of more of the methods described herein. The software may reside entirely in the memory, or may also reside, completely or at least partially, within the RAM and/or within the processor during execution thereof by the computer system. Thus, the memory and the processor also constitute a system comprising machine-readable code.

In alternative embodiments, the machine operates as a standalone device or may be connected, e.g., networked to other machines, in a networked deployment, the machine may operate in the capacity of a server or a client machine in server-client network environment, or as a peer machine in a peer-to-peer or distributed network environment. The machine may be, for example, a computer, a server, a cluster of servers, a cluster of computers, a web appliance, a distributed computing environment, a cloud computing environment, or any machine capable of executing a set of instructions (sequential or otherwise) that specify actions to be taken by that machine. The term “machine” may also be taken to include any collection of machines that individually or jointly execute a set (or multiple sets) of instructions to perform any one or more of the methodologies discussed herein.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. The method according to claim 19, wherein
the first entity is a provider of the item of personal data and the second entity is a seeker of the item of personal data; and
the data transfer process comprises:
establishing and verifying identities of the provider of the item of personal data and the seeker of the item of personal data in dependence upon Public Decentralized Identifiers of the provider and the seeker stored within one or more blockchains; and
transferring the item of personal data from the provider to the seeker independent of the one or more blockchains.

2. The method according to claim 19, wherein
the first entity is a provider of the item of personal data and the second entity is a seeker of the item of personal data; and
the data transfer process comprises:
establishing and verifying identities of the provider of the item of personal data and the seeker of the item of personal data in dependence upon Public Decentralized Identifiers of the provider and the seeker stored within one or more blockchains;
transferring the item of personal data from the provider to the seeker independent of the one or more blockchains; wherein
the transfer of the item of personal data is performed without an identity of the provider being exposed to either the seeker or any third party.

3. The method according to claim 19, wherein
the first entity is an owner of the item of personal data and the second entity is a party seeking to acquire the item of personal data; and
the data transfer process comprises:
establishing a first Decentralized Identifier (DID) of the owner of the item of personal data;
establishing a second DID of the party seeking to acquire the item of personal data;
establishing a secure connection in dependence upon verifying the first DID and the second DID;
transferring from a first wallet associated with the owner of the personal data the item of personal data to a second wallet associated with the party.

4. The method according to claim 3, wherein
the first DID is not stored within a blockchain;
the second DID is stored within the blockchain; and
the transfer from the first wallet to the second wallet is performed without the item of personal data being stored within the blockchain.

5. The method according to claim 3, wherein
the first DID and the second DID are unique.

6. The method according to claim 3, wherein
the first DID and the second DID are uniquely established for each transfer of personal data.

7. The method according to claim 3, wherein
the item of personal data is a predetermined portion of the personal data relating to provider; and
the predetermined portion of the personal data to which the item of personal data relates is established by the provider.

8. The method according to claim 3, wherein
a granularity of the item of personal data is established by the provider.

9. The method according to claim 3, wherein
the transfer of the item of personal data is performed without an identity of the provider being exposed to either the seeker or any third party; and
the first DID does not contain data identifying the provider.

10. The method according to claim 19, wherein
the first entity is an owner of the personal data;
the second entity is a party seeking to acquire the personal data; and
the data transfer process is established by a first process storing and processing non-personal data stored upon a blockchain relating to the first entity and the second entity; and
the actual transfer of the personal data from the first entity to the second entity is performed by a second process which is independent of the blockchain.

11. The method according to claim 19, wherein
the first entity is a holder of the item of personal data;
the second entity is a proof registry; and
the data transfer process further comprises:
generating by an issuer a consent receipt identity and issuing to the holder a consent enablement credential;
establishing whether the holder accepts the consent enablement credential;
upon a positive determination of the holder accepting the consent enablement credential transmitting an initial acknowledgement from the holder to the issuer;
sending to the holder from the issuer a consent proof request in dependence upon receipt of the initial acknowledgement;
establishing whether the holder accepts the consent proof request;
upon a positive determination of the holder accepting the consent proof request transmitting a consent proof presentation from the holder to the issuer;
establishing whether the issuer accepts the consent proof presentation received from the holder;
upon a positive determination of the issuer accepting the consent proof presentation sending proof data to the proof registry from the issuer.

12. The method according to claim 11, wherein
the data sent to the proof registry comprises the consent proof request, the consent proof verification and the consent receipt identity.

13. The method according to claim 11, further comprising
upon a positive determination of the holder accepting the consent enablement credential transmitting the consent enablement credential to the proof registry;
upon a negative determination of the holder accepting the consent enablement credential transmitting the negative determination to the proof registry; and
storing either the consent enablement credential or negative determination within the proof registry.

14. The method according to claim 11, further comprising
upon a positive determination of the holder accepting the consent proof request transmitting the consent proof presentation credential to the proof registry;
upon a negative determination of the holder accepting the consent proof request transmitting the negative determination to the proof registry; and
storing either the consent proof request or negative determination within the proof registry.

15. The method according to claim 19, wherein
The first entity is a holder of the biomarker; and
The second entity is a researcher seeking access to the biomarker of the holder; and
the data transfer process comprises:
establishing a request for the biomarker from the holder of the biomarker to a distributed ledger software application;
generating with the distributed ledger software application shares S_{I}, S_{R}, and S_{H} in dependence upon receipt of the request for the biomarker;
generating with the distributed ledger software application a credential relating to the biomarker requested;
transmitting the biomarker identity and shares S_{R} and S_{H} to the holder;
determining whether the holder accepts the biomarker identity and shares S_{R} and S_{H};
upon a positive determination of the holder accepting the biomarker identity and shares S_{R} and S_{H} transmitting the biomarker identity and share S_{R} to the researcher seeking access to the biomarker of the holder;
generating a consent receipt identity upon receipt of the biomarker identity and share S_{R};
issuing and transmitting a consent enablement credential to the holder from the researcher;
determining whether the holder accepts the consent enablement credential;
upon a positive determination of the holder accepting the consent enablement credential transmitting an initial acknowledgement to the researcher;
upon receipt by the researcher of the initial acknowledgement generating a consent proof request comprising the share S_{R} and transmitting the consent proof request to a proof registry and the holder;
storing the consent proof request within the proof registry;
determining whether the holder accepts the consent proof request;
upon a positive determination of the holder accepting the consent proof request generating a consent proof presentation and transmitting the consent proof presentation to the researcher.

16. The method according to claim 15, further comprising
verifying by the research the consent proof presentation;
upon a positive verification by the researcher sending proof data to the proof registry; and
storing the proof data within the proof registry.

17. The method according to claim 16, wherein
the proof data comprises the consent proof presentation, verification data relating to the verification by the researcher of the consent proof registration; and
the consent proof presentation is absent revealed attributes of the holder.

18. The method according to claim 19, further comprising
establishing a consent process relating to the data transfer process where the consent process is established by an auditor; wherein
the consent process comprises:
receiving by a proof registry a request for consent data from the auditor;
determining upon the proof registry whether to accept the request;
upon a positive determination retrieving the consent data from a database associated with the proof registry;
transmitting a biomarker identity and a share S_{R} from the proof registry to the auditor;
regenerating by the auditor a secret;
transmitting the secret, biomarker identity and share to a distributed ledger software application;
mapping with the distributed ledger software application a share in dependence upon the biomarker identity and retrieving from another database associated with the distributed ledger software application another share S_{I};
reconstructing with the distributed ledger software application a hash in dependence upon the share S_{R} and another share S_{I};
retrieving an identity through a lookup in dependence upon the hash from a further database associated with the distributed ledger software application; and
transmitting the retrieved identity to the auditor; and the biomarker identity relates to the item of personal data.

19. A method comprising:
transferring an item of personal data between a first entity and a second entity with a data transfer process; wherein
the item of personal data relates to a biomarker of the first entity.
